(19) United States
(12) Patent Application Publication
(10) Pub. No.: US 2026/0345057 A1
(43) Pub. Date: Nov. 5, 2026

(54) SYSTEMS AND METHODS FOR IMPROVING BODILY CAVITY MAP ACCURACY

(71) Applicant: KARDIUM INC., Burnaby (CA)

(72) Inventor: Justin Aaron Michael, Vancouver (CA)

(21) Appl. No.: 19/553,716

(22) Filed: Mar. 2, 2026

Related U.S. Application Data
(60) Provisional application No. 63/789,684, filed on Apr. 16, 2025.

(51) Int. Cl. A61B 5/367; A61B 5/343; A61B 18/00; A61B 18/14

(52) U.S. Cl. CPC A61B 5/367; A61B 5/343; A61B 18/1492; A61B 2018/00577

(57) ABSTRACT

Based on a comparison of electrograms recorded at different locations in a bodily cavity, position information originally recorded for the electrograms may be altered, and a graphical representation of a map of at least part of the bodily cavity may be displayed based at least on the altered position information. The graphical representation of the map that is displayed based at least on the altered position information may reduce map distortion compared to a state of the map prior to the altering of the position information.

### CROSS-REFERENCE TO RELATED APPLICATION

This application claims the benefit of U.S. Provisional Application No. 63/789,684 filed Apr. 16, 2025, the entire disclosure of which is hereby incorporated herein by reference.

### TECHNICAL FIELD

Aspects of this disclosure generally are related to systems and methods for improving the accuracy of a bodily cavity map, such systems and methods applicable to medical systems.

### BACKGROUND

Cardiac surgery was initially undertaken using highly invasive open procedures. A sternotomy, which is a type of incision in the center of the chest that separates the sternum was typically employed to allow access to the heart. In the past several decades, more and more cardiac operations are performed using intravascular or percutaneous techniques, where access to inner organs or other tissue is gained via a catheter.

Intravascular or percutaneous surgeries benefit patients by reducing surgery risk, complications and recovery time. However, the use of intravascular or percutaneous technologies also raises some particular challenges. Medical devices used in intravascular or percutaneous surgery need to be deployed via catheter systems which significantly increase the complexity of the device structure. As well, doctors do not have direct visual contact with the medical devices once the devices are positioned within the body.

One example of where intravascular or percutaneous medical techniques have been employed is in the treatment of a heart disorder called atrial fibrillation. Atrial fibrillation is a disorder in which spurious electrical signals cause an irregular heartbeat. Atrial fibrillation has been treated with various methods including a technique known as the “PV isolation” (where “PV” refers to the pulmonary vein). Research has shown that atrial fibrillation typically begins in the pulmonary veins or at the point where they attach to the left atrium. There are typically four major pulmonary veins, and some or all may be a focal point for activity that may cause atrial fibrillation. During this PV isolation procedure, physicians create specific patterns of lesions in the heart to block various paths taken by the spurious electrical signals. The patterns of lesions may include a pattern of one or more lesions that encircles at least one of the pulmonary veins. Lesions were originally created using incisions, but are now typically created by ablating the tissue with various techniques including pulsed field ablation (“PFA”) (also known as irreversible electroporation), radiofrequency (“RF”) ablation, microwave ablation, laser ablation, and cryogenic ablation. Lesion formation may be performed with a high success rate under the direct vision that is provided in open procedures, but is relatively complex to perform intravascularly or percutaneously because of the difficulty in creating the lesions in the correct locations. The continuity, transmurality, and placement of the lesion patterns that are formed can impact the ability to block paths taken within the heart by spurious electrical signals. In this regard, the monitoring of intracardiac electrograms (i.e., cardiac signals measured intracorporeally) may be used to determine the efficacy of the lesion patterns.

To assist in the placement of lesions at correct locations in a bodily cavity, intravascular or percutaneous surgeries may utilize a map-generation system that produces a computerized map of the bodily cavity, which can include or be used with a tracking system that tracks the real-time location of the medical device with respect to the map of the bodily cavity. This real-time location of the medical device with respect to the map of the bodily cavity may be visually presented in a graphical representation, e.g., on a computer monitor, so that the physician can see approximately where the medical device is presently located in the bodily cavity in order to assist the physician in properly placing lesions at correct locations in the bodily cavity.

The present inventor recognized that the accuracy of the map depends at least on the conditions in which spatial locations of the tissue wall in the bodily cavity are determined. In this regard, the present inventor recognized that detected spatial locations of the tissue wall are susceptible to error induced by tissue deformation, such as that caused by the applied force of the catheter contacting the tissue wall. In such cases, the present inventor recognized that the same spatial location detected in different states of tissue deformation may correspond to different portions of tissue, or the same portion of tissue may correspond to different spatial locations.

In this regard, it can be understood that the accuracy of the computer-generated map of the bodily cavity of a patient is difficult to determine, but is quite important, not only for assisting with the accurate placement of lesions in the bodily cavity, but also in any other context where treatment or diagnostic measurement by a catheter is necessary, especially when direct visibility of the catheter is unavailable. Accordingly, a need in the art exists for improving the accuracy of computer-generated bodily cavity maps.

### SUMMARY

At least the above-discussed need is addressed and technical solutions are achieved in the art by various embodiments of the present invention. In some embodiments, a medical system includes a data processing device system; an input-output device system communicatively connected to the data processing device system, the input-output device system communicatively connectable to or connected to a plurality of electrodes located on a catheter of a catheter device system; and a memory device system communicatively connected to the data processing device system and storing a program executable by the data processing device system. In some embodiments, the data processing device system is configured by the program at least to access first electrogram information indicating a first electrogram set; access first position information indicating at least a position associated with at least one electrogram in the first electrogram set; access second electrogram information indicating a second electrogram set; access second position information indicating at least a position associated with at least one electrogram in the second electrogram set, the position associated with the at least one electrogram in the first electrogram set different than the position associated with the at least one electrogram in the second electrogram set; alter, based at least on a comparison of the at least one electrogram in the first electrogram set and the at least one electrogram in the second electrogram set, the first position information, the second position information, or both the first position information and the second position information; and cause, via the input-output device system, display of a visual representation of a map of at least part of a bodily cavity based at least on the altered first position information, the altered second position information, or both the altered first position information and the altered second position information.

In some embodiments, the first position information is associated with a first portion of the bodily cavity, the second position information is associated with a second portion of the bodily cavity, and the data processing device system is configured by the program at least to produce the map at least by stitching at least part of a first submap of a first part of the bodily cavity with at least part of a second submap of a second part of the bodily cavity, the first submap of the first part of the bodily cavity mapping at least part of the first portion of the bodily cavity, and the second submap of the second part of the bodily cavity mapping at least part of the second portion of the bodily cavity. In some embodiments, the stitching utilizes the altered first position information, the altered second position information, or both the altered first position information and the altered second position information.

In some embodiments, the data processing device system is configured by the program at least to determine that the first position information and the second position information are associated with a same tissue region of the bodily cavity, and the altering of the first position information, the second position information, or both the first position information and the second position information produces altered position information to indicate the same tissue region of the bodily cavity.

In some embodiments, the comparison of the at least one electrogram in the first electrogram set and the at least one electrogram in the second electrogram set indicates that the first position information and the second position information are associated with a same tissue region of the bodily cavity. In some embodiments, the altering of the first position information, the second position information, or both the first position information and the second position information produces altered position information to indicate the same tissue region of the bodily cavity. In some embodiments, the altered position information indicates a resulting position associated with both the at least one electrogram in the first electrogram set and the at least one electrogram in the second electrogram set, the resulting position being other than (a) the position associated with the at least one electrogram in the first electrogram set, (b) the position associated with the at least one electrogram in the second electrogram set, or each of (a) and (b), the resulting position indicating a location in the same tissue region of the bodily cavity. In some embodiments, the data processing device system is configured by the program at least to exclude (c) at least part of the first position information, (d) at least part of the second position information, or both (c) and (d) in the altered position information.

In some embodiments, the comparison of the at least one electrogram in the first electrogram set and the at least one electrogram in the second electrogram set is a comparison of a plurality of electrograms in the first electrogram set with a plurality of electrograms in the second electrogram set. In some embodiments, the comparison of the plurality of electrograms in the first electrogram set with the plurality of electrograms in the second electrogram set indicates that the first position information and the second position information are associated with a same tissue region of the bodily cavity. In some embodiments, the altering of the first position information, the second position information, or both the first position information and the second position information produces altered position information to indicate the same tissue region of the bodily cavity.

In some embodiments, the data processing device system is configured by the program at least to receive, via the input-output device system, first navigation information and second navigation information. In some embodiments, the first position information is or is derived from the first navigation information. In some embodiments, the second position information is or is derived from the second navigation information. In some embodiments, the input-output device system is communicatively connected to a catheter navigation system, and the first navigation information and the second navigation information are received, via the input-output device system, from the catheter navigation system.

In some embodiments, the altering of the first position information, the second position information, or both the first position information and the second position information includes altering the at least the position associated with the at least one electrogram in the first electrogram set, altering the at least the position associated with the at least one electrogram in the second electrogram set, or both altering the at least the position associated with the at least one electrogram in the first electrogram set and altering the at least the position associated with the at least one electrogram in the second electrogram set.

In some embodiments, the comparison of the at least one electrogram in the first electrogram set and the at least one electrogram in the second electrogram set includes determining a similarity metric indicating a degree of similarity between the at least one electrogram in the first electrogram set and the at least one electrogram in the second electrogram set. In some embodiments, a higher degree of similarity indicated by the determined similarity metric results in a greater degree of the altering.

In some embodiments, the data processing device system is configured by the program at least to produce the map, and the produced map reduces distortion in a previous version of the map due at least to the altering.

In some embodiments, the input-output device system is communicatively connected to the catheter device system. In some embodiments, the data processing device system is configured by the program at least to receive the first electrogram information from a first electrode set from the plurality of electrodes and to receive the second electrogram information from a second electrode set from the plurality of electrodes. In some embodiments, the first electrode set comprises at least one electrode that is not in the second electrode set. In some embodiments, at least some of the plurality of electrodes are configured to cause pulsed field ablation of tissue. In some embodiments, at least one electrode from the first electrode set, the second electrode set, or both the first electrode set and the second electrode set is or are configured to cause pulsed field ablation of tissue.

In some embodiments, the bodily cavity is a heart.

In some embodiments, the position associated with the at least one electrogram in the first electrogram set is different than the position associated with the at least one electrogram in the second electrogram set.

In some embodiments, the first electrogram information indicates first electrophysiological information recorded by the catheter device system at a first position in the bodily cavity, and the second electrogram information indicates second electrophysiological information recorded by the catheter device system at a second position in the bodily cavity, the second position different than the first position.

In some embodiments, each electrogram in the first electrogram set and each electrogram in the second electrogram set is a unipolar electrogram.

In some embodiments, each electrogram in the first electrogram set and each electrogram in the second electrogram set is a bipolar or omnipolar electrogram.

Various embodiments of the present invention may include systems, devices, or machines that are or include combinations or subsets of any one or more of the systems, devices, or machines and associated features thereof summarized above or otherwise described herein (which should be deemed to include the figures).

Further, all or part of any one or more of the systems, devices, or machines summarized above or otherwise described herein or combinations or sub-combinations thereof may implement or execute all or part of any one or more of the processes or methods described herein or combinations or sub-combinations thereof.

For example, in some embodiments, a method is executed by a data processing device system according to a program stored by a communicatively connected memory device system, the data processing device system also communicatively connected to an input-output device system, the input-output device system communicatively connected to a plurality of electrodes located on a catheter of a catheter device system, and the method including: accessing first electrogram information indicating a first electrogram set; accessing first position information indicating at least a position associated with at least one electrogram in the first electrogram set; accessing second electrogram information indicating a second electrogram set; accessing second position information indicating at least a position associated with at least one electrogram in the second electrogram set, the position associated with the at least one electrogram in the first electrogram set different than the position associated with the at least one electrogram in the second electrogram set; altering, based at least on a comparison of the at least one electrogram in the first electrogram set and the at least one electrogram in the second electrogram set, the first position information, the second position information, or both the first position information and the second position information; and causing, via the input-output device system, display of a visual representation of a map of at least part of a bodily cavity based at least on the altered first position information, the altered second position information, or both the altered first position information and the altered second position information.

It should be noted that various embodiments of the present invention include variations of the methods or processes summarized above or otherwise described herein (which should be deemed to include the figures) and, accordingly, are not limited to the actions described or shown in the figures or their ordering, and not all actions shown or described are required according to various embodiments. According to various embodiments, such methods may include more or fewer actions and different orderings of actions. Any of the features of all or part of any one or more of the methods or processes summarized above or otherwise described herein may be combined with any of the other features of all or part of any one or more of the methods or processes summarized above or otherwise described herein.

In addition, a computer program product may be provided that includes program code portions for performing some or all of any one or more of the methods or processes and associated features thereof described herein, when the computer program product is executed by a computer or other computing device or device system. Such a computer program product may be stored on one or more computer-readable storage mediums, also referred to as one or more computer-readable data storage mediums or a computer-readable storage medium system.

For example, in some embodiments, one or more computer-readable storage mediums store a program executable by a data processing device system communicatively connected to an input-output device system, the input-output device system communicatively connectable to a plurality of electrodes located on a catheter of a catheter device system. In some embodiments, the program includes: first access instructions configured to cause accessing of first electrogram information indicating a first electrogram set; second access instructions configured to cause accessing of first position information indicating at least a position associated with at least one electrogram in the first electrogram set; third access instructions configured to cause accessing of second electrogram information indicating a second electrogram set; fourth access instructions configured to cause accessing of second position information indicating at least a position associated with at least one electrogram in the second electrogram set, the position associated with the at least one electrogram in the first electrogram set different than the position associated with the at least one electrogram in the second electrogram set; alteration instructions configured to cause altering, based at least on a comparison of the at least one electrogram in the first electrogram set and the at least one electrogram in the second electrogram set, of the first position information, the second position information, or both the first position information and the second position information; and display instructions configured to cause, via the input-output device system, display of a visual representation of a map of at least part of a bodily cavity based at least on the altered first position information, the altered second position information, or both the altered first position information and the altered second position information.

In some embodiments, each of any of one or more or all of the computer-readable data storage mediums or medium systems (also referred to as processor-accessible memory device systems) described herein is a non-transitory computer-readable (or processor-accessible) data storage medium or medium system (or memory device system) including or consisting of one or more non-transitory computer-readable (or processor-accessible) storage mediums (or memory devices) storing the respective program(s) which may configure a data processing device system to execute some or all of any of one or more of the methods or processes described herein.

Further, any of all or part of one or more of the methods or processes and associated features thereof discussed herein may be implemented or executed on or by all or part of a device system, apparatus, or machine, such as all or a part of any of one or more of the systems, apparatuses, or machines described herein or a combination or sub-combination thereof.

### BRIEF DESCRIPTION OF THE DRAWINGS

It is to be understood that the attached drawings are for purposes of illustrating aspects of various embodiments and may include elements that are not to scale.

FIG. 1 includes a schematic representation of a transducer-activation system according to various example embodiments, the transducer-activation system including a data processing device system, an input-output device system, and a memory device system.

FIG. 2 includes a cutaway diagram of a heart showing a transducer-based device percutaneously placed in a left atrium of the heart, according to various example embodiments.

FIG. 3A includes a partially schematic representation of a medical system according to various example embodiments, the medical system including a data processing device system, an input-output device system, a memory device system, and a transducer-based device including a plurality of transducers and an expandable structure shown in a delivery or unexpanded configuration.

FIG. 3B includes the representation of the medical system of FIG. 3A with the expandable structure shown in a deployed or expanded configuration, according to some embodiments.

FIG. 4 includes a schematic representation of a transducer-based device that includes a flexible circuit structure, according to various example embodiments.

FIG. 5 includes a block diagram of various methods for improving the accuracy of a bodily cavity map, according to some embodiments.

FIG. 6 illustrates a graphical representation of a heart cavity, transducers, and electrograms, which may be visually presented under control of at least a data processing device system, according to various embodiments.

FIG. 7 illustrates a portion of a map of a bodily cavity with overlaid electrograms associated with respective locations on the map, the map including distortion, according to various embodiments, where the overlaid electrograms may or may not be included as part of the map itself, according to various embodiments.

FIG. 8 illustrates an altered portion of the map of the bodily cavity of FIG. 7, the altering reducing the distortion of the map of FIG. 7, and the altering based at least on a comparison of electrograms associated with the map of the bodily cavity of FIG. 7, according to various embodiments.

FIGS. 9A and 9B illustrates one type of situation in which distortion in a map of a bodily cavity may arise, such type of situation involving stretching of a tissue wall of the bodily cavity due to pressing of the medical device inside the bodily cavity, according to various embodiments.

### DETAILED DESCRIPTION

The above-discussed need in the art is addressed and technical solutions are achieved according to various embodiments of the present invention. In some embodiments, improved bodily cavity map accuracy is achieved at least by altering position information associated with a map of a bodily cavity based at least on a comparison of electrograms associated with various map positions. In some embodiments, the comparison of electrograms may indicate a region of distortion in a map of a bodily cavity, and the altering may reduce such distortion and produce a more accurate map of the bodily cavity. In some embodiments, the comparison of electrograms includes determining a degree of similarity between electrograms indicated as supposedly being associated with different locations (e.g., in three-dimensional space in some embodiments). In some embodiments, a high degree of similarity between the electrograms indicates that the supposed different locations are actually the same locations (or at least are closer than previously supposed), which may be corrected by altering either or both of the associated supposed different locations.

It should be noted that various embodiments of the invention are not limited to these features and benefits, which are referred to for purposes of illustration only, and additional and alternative features and benefits will become apparent from the following description in conjunction with reference to the figures.

In this regard, in the following description, certain specific details are set forth in order to provide a thorough understanding of various embodiments of the invention. However, one skilled in the art will understand that the invention may be practiced at a more general level without these details. In other instances, well-known structures have not been shown or described in detail to avoid unnecessarily obscuring descriptions of various embodiments of the invention.

Any reference throughout this specification to “one embodiment”, “an embodiment”, “an example embodiment”, “an illustrated embodiment”, “a particular embodiment”, and the like means that a particular feature, structure or characteristic described in connection with the embodiment is included in at least one embodiment. Thus, any appearance of the phrase “in one embodiment”, “in an embodiment”, “in an example embodiment”, “in this illustrated embodiment”, “in this particular embodiment”, or the like in this specification is not necessarily always referring to one embodiment or a same embodiment. Furthermore, the particular features, structures, or characteristics of different embodiments may be combined in any suitable manner to form one or more other embodiments. In one embodiment, all references to “some embodiments” may refer to the same single embodiment.

Unless otherwise explicitly noted or required by context, the word “or” is used in this disclosure in a non-exclusive sense. In addition, unless otherwise explicitly noted or required by context, the word “set” is intended to mean one or more. For example, the phrase “a set of objects” means one or more of the objects. In some embodiments, the word “subset” is intended to mean a set having the same or fewer elements of those present in the subset's parent or superset. In other embodiments, the word “subset” is intended to mean a set having fewer elements of those present in the subset's parent or superset. In this regard, when the word “subset” is used, some embodiments of the present invention utilize the meaning that “subset” has the same or fewer elements of those present in the subset's parent or superset, and other embodiments of the present invention utilize the meaning that “subset” has fewer elements of those present in the subset's parent or superset.

Further, the phrase “at least” is or may be used herein at times merely to emphasize the possibility that other elements may exist besides those explicitly listed. However, unless otherwise explicitly noted (such as by the use of the term “only”) or required by context, non-usage herein of the phrase “at least” nonetheless includes the possibility that other elements may exist besides those explicitly listed. For example, the phrase ‘based at least on A’ includes A as well as the possibility of one or more other additional elements besides A. In the same manner, the phrase ‘based on A’ includes A, as well as the possibility of one or more other additional elements besides A. However, the phrase ‘based only on A’ includes only A. Similarly, the phrase ‘configured at least to A’ includes a configuration to perform A, as well as the possibility of one or more other additional actions besides A. In the same manner, the phrase ‘configured to A’ includes a configuration to perform A, as well as the possibility of one or more other additional actions besides A. However, the phrase ‘configured only to A’ means a configuration to perform only A.

The word “device”, the word “machine”, the word “system”, and the phrase “device system” all are intended to include one or more physical devices or sub-devices (e.g., pieces of equipment) that interact to perform one or more functions, regardless of whether such devices or sub-devices are located within a same housing or different housings. However, it may be explicitly specified according to various embodiments that a device or machine or device system resides entirely within a same housing to exclude embodiments where the respective device, machine, system, or device system resides across different housings. The word “device” may equivalently be referred to as a “device system” in some embodiments, and the word “system” may equivalently be referred to as a “device system” in some embodiments.

Further, the phrase “in response to” may be used in this disclosure. For example, this phrase may be used in the following context, where an event A occurs in response to the occurrence of an event B. In this regard, such phrase includes, for example, that at least the occurrence of the event B causes or triggers or is a necessary precondition for the event A, according to various embodiments.

In some embodiments, the word “adjacent”, the word “proximate”, and the like refer at least to a sufficient closeness between the objects or events defined as adjacent, proximate, or the like, to allow the objects or events to interact in a designated way. For example, in the case of physical objects, if object A performs an action on an adjacent or proximate object B, objects A and B would have at least a sufficient closeness to allow object A to perform the action on object B. In this regard, some actions may require contact between the associated objects, such that if object A performs such an action on an adjacent or proximate object B, objects A and B would be in contact, for example, in some instances or embodiments where object A needs to be in contact with object B to successfully perform the action. In some embodiments, the word “adjacent”, the word “proximate”, and the like additionally or alternatively refer to objects or events that do not have another substantially similar object or event between them. For example, object or event A and object or event B could be considered adjacent or proximate (e.g., physically or temporally) if they are immediately next to each other (with no other object or event between them) or are not immediately next to each other but no other object or event that is substantially similar to object or event A, object or event B, or both objects or events A and B, depending on the embodiment, is between them. In the context of electrodes, discussed at various points in this description, two electrodes may be considered adjacent, in some embodiments, when the two electrodes have no other electrodes between them that is the same in function to any of the two electrodes (e.g., the electrodes are tissue ablation electrodes). In some embodiments, the word “adjacent”, the word “proximate”, and the like additionally or alternatively refer to at least a sufficient closeness between the objects or events defined as adjacent, proximate, and the like, the sufficient closeness being within a range that does not place any one or more of the objects or events into a different or dissimilar region or time period, or does not change an intended function of any one or more of the objects or events or of an encompassing object or event that includes a set of the objects or events. Different embodiments of the present invention adopt different ones or combinations of the above definitions. Of course, however, the word “adjacent”, the word “proximate”, and the like are not limited to any of the above example definitions, according to some embodiments. In addition, the word “adjacent” and the word “proximate” do not have the same definition, according to some embodiments.

The word “ablation” as used in this disclosure should be understood to include, for example, any disruption to certain properties of tissue. Most commonly, the disruption is to the electrical conductivity and is achieved by heating, which can be generated with resistive or radiofrequency (RF) techniques for example. However, any other technique for such disruption may be included when the word “ablation” is used, such as mechanical, chemical, electroporation (e.g., pulsed field ablation), or optical techniques. Various catheters described in this disclosure, may in some embodiments, be employed to deliver ablative energy.

According to some embodiments, the word “fluid” as may be used in this disclosure should be understood to include any fluid that can be contained within a bodily cavity or can flow into or out of, or both into and out of a bodily cavity via one or more bodily openings positioned in fluid communication with the bodily cavity. In the case of cardiac applications, fluid such as blood will flow into and out of various intracardiac cavities (e.g., a left atrium or a right atrium).

According to some embodiments, the words “bodily opening” as may be used in this disclosure should be understood to include a naturally occurring bodily opening or channel or lumen; a bodily opening or channel or lumen formed by an instrument or tool using techniques that can include, but are not limited to, mechanical, thermal, electrical, chemical, and exposure or illumination techniques; a bodily opening or channel or lumen formed by trauma to a body; or various combinations of one or more of the above. Various elements having respective openings, lumens, or channels and positioned within the bodily opening (e.g., a catheter sheath) may be present in various embodiments. These elements may provide a passageway through a bodily opening for various devices employed in various embodiments.

The words “bodily cavity” as used in this disclosure should be understood to mean a cavity in a body, in some embodiments. The bodily cavity may be a cavity or chamber provided in a bodily organ (e.g., an intracardiac cavity of a heart). In some embodiments, the bodily cavity is a heart cavity.

The word “tissue” as used in some embodiments in this disclosure should be understood to include any surface-forming tissue that is used to form a surface of a body or a surface within a bodily cavity, a surface of an anatomical feature or a surface of a feature associated with a bodily opening positioned in fluid communication with the bodily cavity. The tissue can include part, or all, of a tissue wall or membrane that defines a surface of the bodily cavity. In this regard, the tissue can form an interior surface of the cavity that surrounds a fluid within the cavity. In the case of cardiac applications, “tissue” can include tissue used to form an interior surface of an intracardiac cavity such as a left atrium or a right atrium.

According to some embodiments, the term “transducer” as used in this disclosure should be interpreted broadly as any device capable of transmitting or delivering energy (e.g., such as tissue ablative energy), distinguishing between fluid and tissue, sensing or sampling information, sensing or sampling temperature, creating heat, ablating tissue, sensing, sampling or measuring electrical activity of a tissue surface (e.g., sensing, sampling or measuring intracardiac electrograms, or sensing, sampling or measuring intracardiac voltage data), stimulating tissue, providing location information (e.g., in conjunction with a navigation system), or any combination thereof.

In some embodiments, these functions of transducers are performed by electrodes included in the transducers. In some embodiments, the electrodes are independent of any over-arching transducer structure. In some embodiments, the same electrode(s) that are configured to sample or sense may also be configured to deliver, e.g., tissue ablative energy. In some embodiments, a first transducer or electrode set may be configured to deliver, e.g., tissue ablative energy, and a sensor set (which may be a second transducer or electrode set in some embodiments) that is mutually exclusive with the first electrode set may be configured to sample or sense (e.g., sampling or sensing electrophysiological activity from which one or more electrograms may be derived). In some embodiments, the first transducer or electrode set includes at least some transducers or electrodes in the second transducer or electrode set.

A transducer may convert input energy of one form into output energy of another form. At least in this regard, an electrode may itself be considered a transducer, or form part thereof.

Without limitation, a transducer may include an electrode that is configured or functions as, or as part of, a sensing or sampling device included in the transducer, an energy delivery device included in the transducer, or both a sensing or sampling device and an energy delivery device included in the transducer. Without limitation, an electrode may be configured or function as, or as part of, a sensing or sampling device independent of any over-arching transducer structure, an energy delivery device independent of any over-arching transducer structure, or both a sensing or sampling device and an energy delivery device independent of any over-arching transducer structure.

In some embodiments, each of some or all sensing or sampling transducers may be configured as a thermocouple, thermistor, or resistance temperature detector (“RTD”).

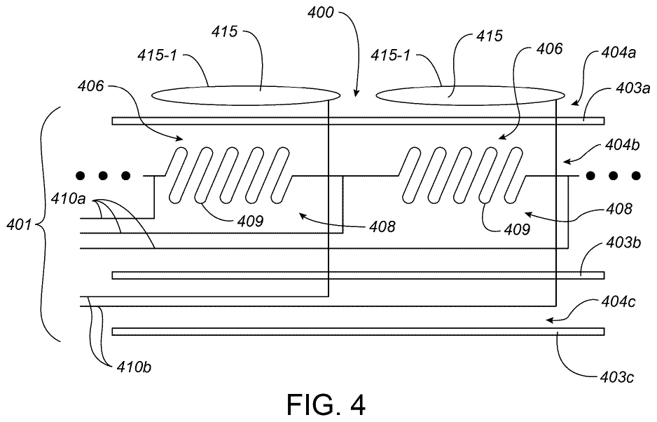

A transducer may be constructed from several parts, which may be discrete components or may be integrally formed. In this regard, although transducers, electrodes, or both transducers and electrodes are referenced with respect to various embodiments, it is understood that other transducers or transducer elements may be employed in other embodiments. It is understood that a reference to a particular transducer in various embodiments may also imply a reference to an electrode, as an electrode may be part of the transducer as shown, e.g., at least with FIG. 4 discussed below. In some embodiments, references to transducers may be replaced with references to electrodes, as, e.g., not all components described with respect to transducers herein, such as those shown, e.g., at least with FIG. 4 discussed below, are needed in all applications. In some embodiments, an electrode itself may be considered an energy transmitting transducer (for example, when an electrode is employed to transmit tissue ablation energy).

The term “activation” as may be used in this disclosure, according to some embodiments, should be interpreted broadly as making active a particular function as related to various transducers or electrodes disclosed in this disclosure. Particular functions may include, but are not limited to, tissue ablation (e.g., PFA or thermal ablation such as RF ablation), sensing, sampling, or measuring electrophysiological activity (e.g., sensing, sampling, or measuring intracardiac electrogram information, or sensing, sampling, or measuring intracardiac voltage data), sensing, sampling, or measuring temperature, and sensing, sampling, or measuring electrical characteristics (e.g., tissue impedance or tissue conductivity). For example, in some embodiments, activation of a tissue ablation function of a particular transducer or electrode is initiated by causing energy sufficient for tissue ablation from an energy source device system to be delivered to the particular transducer or electrode. Also, in this example, the activation can last for a duration of time concluding when the ablation function is no longer active, such as when energy sufficient for the tissue ablation is no longer provided to the particular transducer or electrode. In some contexts and embodiments, however, the word “activation” can merely refer to the initiation of the activating of a particular function, as opposed to referring to both the initiation of the activating of the particular function and the subsequent duration in which the particular function is active. In these contexts, the phrase or a phrase similar to “activation initiation” may be used.

The term “electrogram” as used in this disclosure refers to, in some embodiments, a display of information indicating the electrical potentials of a tissue (e.g., the heart) at localized positions within the body. Typically, electrograms are recorded by transducers or electrodes placed directly within a body instead of on an exterior surface of the body. According to various embodiments, the recorded electrograms are graphically displayed in a graphical representation. For example, an electrogram may be displayed as a signal or tracing representing the electrical potentials within the body. In cardiac electrophysiological studies, various electrogram recordings (e.g., unipolar, bipolar, or omnipolar) are used to identify the sequence of activation of electrophysiological activity within the heart. Such recordings can lead to the identification of an arrhythmia circuit which can then be treated by ablation, according to some embodiments.

The term “electrocardiogram” as may be used in this disclosure refers to, in some embodiments, a display of information providing an interpretation of the electrical activity of the heart over a time period. Electrocardiograms are typically detected by electrodes attached to an external or skin-based surface of the body. In this regard, unlike electrograms, electrocardiograms are typically generated transthoracically (i.e., across the thorax or chest).

In the following description, some embodiments of the present invention may be implemented at least in part by a data processing device system, or a controller system, configured by a software program. Such a program may equivalently be implemented as multiple programs, and some, or all, of such software program(s) may be equivalently constructed in hardware. In this regard, reference to “a program” should be interpreted to include one or more programs.

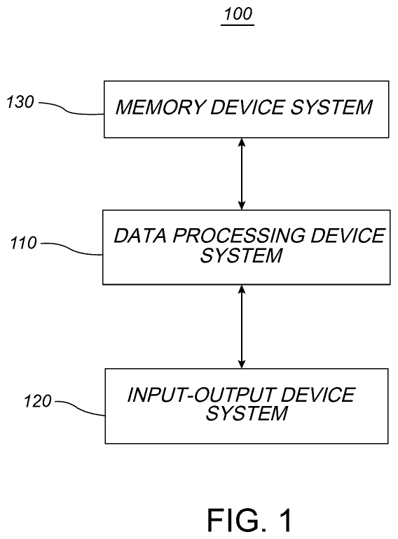

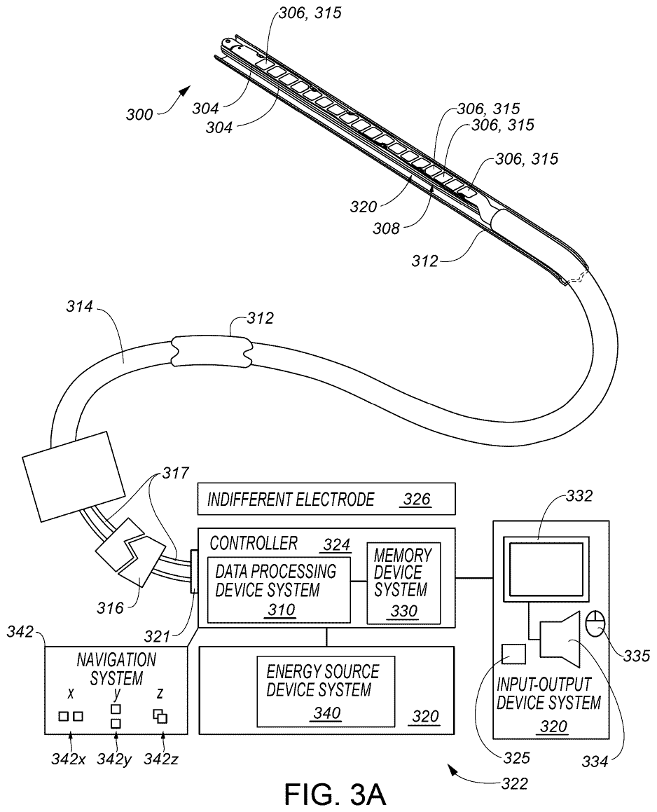

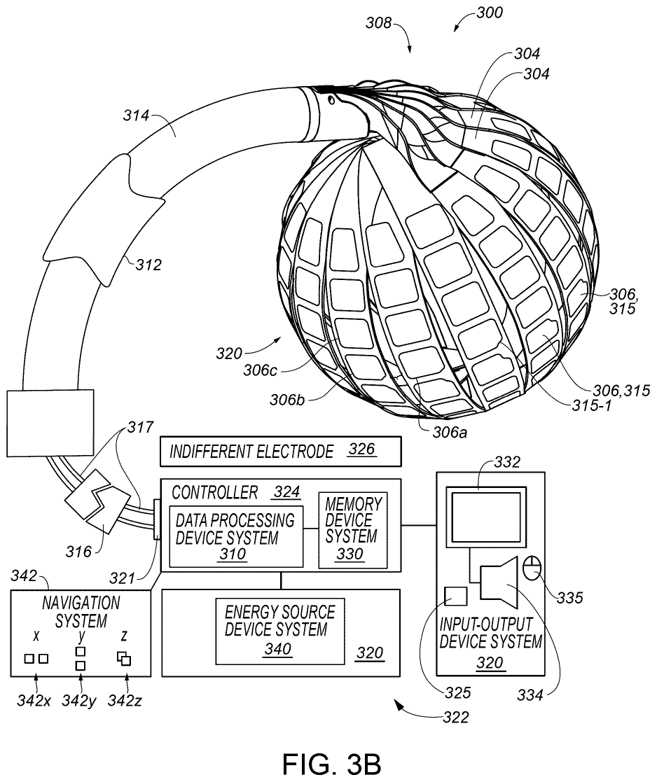

According to some embodiments, the term “program” in this disclosure should be interpreted to include one or more programs including a set of instructions or modules that can be executed by one or more components in a system, such as a controller system or a data processing device system, in order to cause the system to perform one or more operations. The set of instructions or modules may be stored by any kind of memory device, such as those described subsequently with respect to the memory device system 130, 330, or both, shown in at least FIGS. 1 and 3. In addition, this disclosure may describe or similarly describe that the instructions or modules of a program are configured to cause the performance of an action. The phrase “configured to” in this context is intended to include, for example, at least (a) instructions or modules that are presently in a form executable by one or more data processing devices to cause performance of the action (e.g., in the case where the instructions or modules are in a compiled and unencrypted form ready for execution), and (b) instructions or modules that are presently in a form not executable by the one or more data processing devices, but that could be translated into the form executable by the one or more data processing devices to cause performance of the action (e.g., in the case where the instructions or modules are encrypted in a non-executable manner, but through performance of a decryption process, would be translated into a form ready for execution). Such descriptions should be deemed to be equivalent to describing that the instructions or modules are configured to cause the performance of the action. The word “module” may be defined as a set of instructions. In some instances, this disclosure describes that the instructions or modules of a program perform a function. The word “program” and the word “module” may each be interpreted to include multiple sub-programs or multiple sub-modules, respectively. In this regard, reference to a program or a module may be considered to refer to multiple programs or multiple modules.

Further, it is understood that information or data may be operated upon, manipulated, or converted into different forms as it moves through various devices or workflows. In this regard, unless otherwise explicitly noted or required by context, it is intended that any reference herein to information or data or the like includes modifications to that information or data. For example, “data X” may be encrypted for transmission, and a reference to “data X” is intended to include both its encrypted and unencrypted forms, unless otherwise required or indicated by context. For another example, “image information Y” may undergo a noise filtering process, and a reference to “image information Y” is intended to include both the pre-processed form and the noise-filtered form, unless otherwise required or indicated by context. In other words, both the pre-processed form and the noise-filtered form are considered to be “image information Y”, unless otherwise required or indicated by context. In order to stress this point, the phrase “or a derivative thereof” or the like may be used herein. Continuing the preceding example, the phrase “image information Y or a derivative thereof” refers to both the pre-processed form and the noise-filtered form of “image information Y”, unless otherwise required or indicated by context, with the noise-filtered form potentially being considered a derivative of “image information Y”. However, non-usage of the phrase “or a derivative thereof” or the like nonetheless includes derivatives or modifications of information or data unless otherwise explicitly noted or required by context.

In some embodiments, the phrase “graphical representation” used herein is intended to include a visual representation presented via a display device system and may include computer-generated text, graphics, animations, or one or more combinations thereof, which may include one or more visual representations originally generated, at least in part, by an image-capture device, such as computerized tomography (“CT”) scan images, magnetic resonance imaging (“MRI”) images, or maps created from a catheter navigation system (e.g., electropotential navigation system or an electromagnetic navigation system), according to some embodiments. The graphical representation may include various entities depicted in a two-dimensional manner, in some embodiments. The graphical representation may include various entities depicted in a three-dimensional manner, in some embodiments. The graphical representation may include various entities depicted in a two-dimensional manner that are mapped from a three-dimensional space into a two-dimensional coordinate system, in some embodiments. The graphical representation may include other information, including recorded data, such as electrograms.

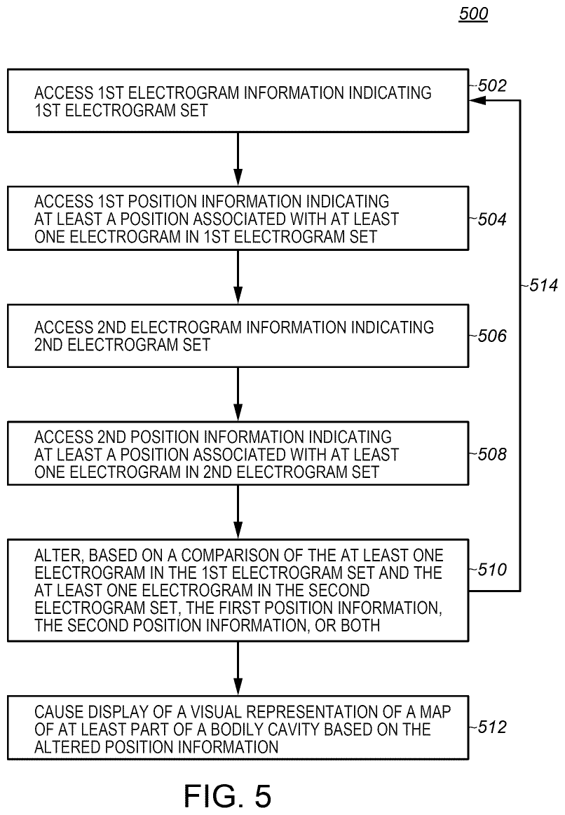

Example methods are described herein with respect to FIG. 5. FIG. 5 includes blocks associated with actions, computer-executable instructions, or both, according to various embodiments. It should be noted that the respective instructions associated with any such blocks therein need not be separate instructions and may be combined with other instructions to form a combined instruction set. The same set of instructions may be associated with more than one block. In this regard, the block arrangement shown in each of the method figures herein is not limited to an actual structure of any program or set of instructions or required ordering of method tasks, and such method figures, according to some embodiments, merely illustrate the tasks that instructions are configured to perform, for example, upon execution by a data processing device system in conjunction with interactions with one or more other devices or device systems.

Each of the phrases “derived from” or “derivation of” or “derivation thereof” or the like may be used herein, according to some embodiments, to mean to come from at least some part of a source, be created from at least some part of a source, or be developed as a result of a process in which at least some part of a source forms an input, according to various embodiments. For example, a data set derived from some particular portion of data may include at least some part of the particular portion of data, or may be created from at least part of the particular portion of data, or may be developed in response to a data manipulation process in which at least part of the particular portion of data forms an input. In some embodiments, a data set may be derived from a subset of the particular portion of data. In some embodiments, the particular portion of data is analyzed to identify a particular subset of the particular portion of data, and a data set is derived from the subset. In various ones of these embodiments, the subset may include some, but not all, of the particular portion of data. In some embodiments, changes in at least one part of a particular portion of data may result in changes in a data set derived at least in part from the particular portion of data.

In this regard, each of the phrases “derived from” or “derivation of” or “derivation thereof” or the like may be used herein merely to emphasize the possibility that such data or information may be modified or subject to one or more operations. For example, if a device generates first data for display, the process of converting the generated first data into a format capable of being displayed may alter the first data. This altered form of the first data may be considered a derivative or derivation of the first data. For instance, the first data may be a one-dimensional array of numbers, but the display of the first data may be a color-coded bar chart representing the numbers in the array. For another example, if the above-mentioned first data is transmitted over a network, the process of converting the first data into a format acceptable for network transmission or understanding by a receiving device may alter the first data. As before, this altered form of the first data may be considered a derivative or derivation of the first data. For yet another example, generated first data may undergo a mathematical operation, a scaling, or a combining with other data to generate other data that may be considered derived from the first data. In this regard, it can be seen that data is commonly changing in form or being combined with other data throughout its movement through one or more data processing device systems, and any reference to information or data herein is intended in some embodiments to include these and like changes, regardless of whether or not the phrase “derived from” or “derivation of” or “derivation thereof” or the like is used in reference to the information or data. As indicated above, usage of the phrase “derived from” or “derivation of” or “derivation thereof” or the like merely emphasizes the possibility of such changes. Accordingly, in some embodiments, the usage, non-usage, addition of, or deletion of the phrase “derived from” or “derivation of” or “derivation thereof” or the like should have no impact on the interpretation of the respective data or information. For example, the above-discussed color-coded bar chart may be considered a derivative of the respective first data or may be considered the respective first data itself, whether or not the phrase “derived from” or “derivation of” or “derivation thereof” or the like is used, according to some embodiments.

FIG. 1 schematically illustrates a portion of a transducer-activation system or controller system thereof 100 that may be employed to at least select, control, activate, or monitor a function or activation of a number of electrodes or transducers (e.g., ablation transducers configured to cause thermal ablation or ablation transducers configured to cause PFA), according to some embodiments. In some embodiments in which the system 100 is configured at least to transmit pulsed field ablation energy, such system 100 may be considered at least part of a pulsed field ablation system. The system 100 includes a data processing device system 110, an input-output device system 120, and a processor-accessible memory device system 130. The processor-accessible memory device system 130 and the input-output device system 120 are communicatively connected to the data processing device system 110. According to some embodiments, various components such as data processing device system 110, input-output device system 120, and processor-accessible memory device system 130 form at least part of a controller system (e.g., controller system 324 shown in FIG. 3).

The data processing device system 110 includes one or more data processing devices that implement or execute, in conjunction with other devices, such as those in the system 100, various methods and functions described herein, including those described with respect to methods exemplified in FIG. 5. Each of the phrases “data processing device”, “data processor”, “processor”, “controller”, “computing device”, “computer” and the like is intended to include any data or information processing device, such as a central processing unit (CPU), a control circuit, a desktop computer, a laptop computer, a mainframe computer, a tablet computer, a personal digital assistant, a cellular or smart phone, and any other device for processing data, managing data, or handling data, whether implemented with electrical, magnetic, optical, quantum, or biological components, or otherwise.

The memory device system 130 includes one or more processor-accessible memory devices configured to store one or more programs and information, including the program(s) and information needed to execute the methods or functions described herein, including those described with respect to FIG. 5. The memory device system 130 may be a distributed processor-accessible memory device system including multiple processor-accessible memory devices communicatively connected to the data processing device system 110 via a plurality of computers and/or devices. However, the memory device system 130 need not be a distributed processor-accessible memory system and, consequently, may include one or more processor-accessible memory devices located within a single data processing device or housing.

Each of the phrases “processor-accessible memory” and “processor-accessible memory device” and the like is intended to include any processor-accessible data storage device or medium, whether volatile or nonvolatile, electronic, magnetic, optical, or otherwise, including but not limited to, registers, hard disk drives, Compact Discs, DVDs, flash memories, ROMs, and RAMs. In some embodiments, each of the phrases “processor-accessible memory” and “processor-accessible memory device” is intended to include or be a processor-accessible (or computer-readable) data storage medium. In some embodiments, each of the phrases “processor-accessible memory” and “processor-accessible memory device” may include or may be a non-transitory processor-accessible (or computer-readable) data storage medium. In some embodiments, the processor-accessible memory device system 130 may include or may be a non-transitory processor-accessible (or computer-readable) data storage medium system. In some embodiments, the memory device system 130 may include or may be a non-transitory processor-accessible (or computer-readable) storage medium system or data storage medium system including or consisting of one or more non-transitory processor-accessible (or computer-readable) storage or data storage mediums.

The phrase “communicatively connected” is intended to include any type of connection, whether wired or wireless, between devices, data processors, or programs between which data may be communicated. Further, the phrase “communicatively connected” is intended to include a connection between devices or programs within a single data processor or computer, a connection between devices or programs located in different data processors or computers, and a connection between devices not located in data processors or computers at all. In this regard, although the memory device system 130 is shown separately from the data processing device system 110 and the input-output device system 120, one skilled in the art will appreciate that the memory device system 130 may be located completely or partially within the data processing device system 110 or the input-output device system 120. Further in this regard, although the input-output device system 120 is shown separately from the data processing device system 110 and the memory device system 130, one skilled in the art will appreciate that such system may be located completely or partially within the data processing system 110 or the memory device system 130, for example, depending upon the contents of the input-output device system 120. Further still, the data processing device system 110, the input-output device system 120, and the memory device system 130 may be located entirely within the same device or housing or may be separately located, but communicatively connected, among different devices or housings. In the case where the data processing device system 110, the input-output device system 120, and the memory device system 130 are located within the same device, the system 100 of FIG. 1 may be implemented by a single application-specific integrated circuit (ASIC) in some embodiments.

The input-output device system 120 may include a mouse, a keyboard, a touch screen, another computer, a processor-accessible memory device system, a network-interface card or network-interface circuitry, or any device or combination of devices from which a desired selection, desired information, instructions, or any other data is input to the data processing device system 110. The input-output device system 120 may include a user-activatable control system that is responsive to a user action. The user-activatable control system may include at least one control element that may be activated or deactivated on the basis of a particular user action. The input-output device system 120 may include any suitable interface for receiving information, instructions or any data from other devices and systems described in various ones of the embodiments. In this regard, the input-output device system 120 may include various ones of other systems described in various embodiments. For example, the input-output device system 120 may include at least a portion of a transducer-based device. The phrase “transducer-based device” or “transducer-based device system” is intended to include one or more physical systems that include various transducers. A tissue ablation (e.g., PFA or thermal) device system that includes one or more transducers may be considered a transducer-based device or device system, according to some embodiments. An electrophysiological activity sensing device system that includes one or more transducers configured to sense electrophysiological activity may be considered a transducer-based device or device system, according to some embodiments. A catheter location tracking system that includes one or more transducers and is configured to track locations or positions of such one or more transducers may be considered a transducer-based device or device system, according to some embodiments. The input-output device system 120 also may include an image generating device system, a display device system, a speaker or audio output device system, a computer, a processor-accessible memory device system, a network-interface card or network-interface circuitry, or any device or combination of devices to which information, instructions, or any other data is output by the data processing device system 110. In this regard, the input-output device system 120 may include various other devices or systems described in various embodiments. The input-output device system 120 may include any suitable interface for outputting information, instructions, or data to other devices and systems described in various ones of the embodiments. If the input-output device system 120 includes a processor-accessible memory device, such memory device may, or may not, form part, or all, of the memory device system 130. The input-output device system 120 may include any suitable interface for outputting information, instructions, or data to other devices and systems described in various ones of the embodiments. In some embodiments, the input-output device system 120 may include a transducer-based device, as discussed above, and in some embodiments, the transducer-based device may act as a device or device system that provides information to, receives instructions or energy from, or both provides information to and receives instructions or energy from the data processing device system 110. In this regard, the input-output device system 120 may include various devices or systems described in various embodiments.

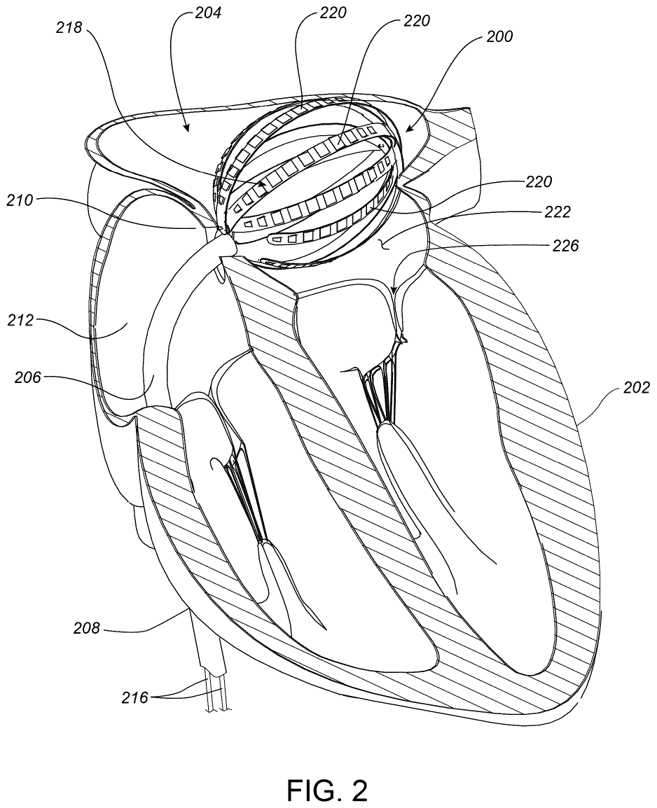

Various embodiments of transducer-based devices are described herein in this disclosure. Some of the described devices are percutaneously or intravascularly deployed. Some of the described devices are moveable between a delivery or unexpanded configuration (e.g., FIG. 3A discussed below) in which a portion of the device is sized for passage through a bodily opening leading to a bodily cavity, and an expanded or deployed configuration (e.g., FIGS. 2 and 3B discussed below) in which the portion of the device has a size too large for passage through the bodily opening leading to the bodily cavity. An example of an expanded or deployed configuration, in some embodiments, is when the portion of the transducer-based device is in its intended-deployed-operational state, which may be inside the bodily cavity when, e.g., performing an intended therapeutic or diagnostic procedure for a patient, or which may be outside the bodily cavity when, e.g., performing testing, quality control, or other evaluation of the device. Another example of the expanded or deployed configuration, in some embodiments, is when the portion of the transducer-based device is being changed from the delivery configuration to the intended-deployed-operational state to a point where the portion of the device now has a size too large for passage through the bodily opening leading to the bodily cavity.

In some example embodiments, the device includes transducers that sample or sense characteristics (e.g., convective cooling, permittivity, force) that distinguish between fluid, such as a fluidic tissue (e.g., blood), and tissue forming an interior surface of the bodily cavity. Such sampled or sensed characteristics can allow a medical system to map the cavity, for example, using positions of openings or ports into and out of the cavity to determine a position or orientation (e.g., pose), or both, of the portion of the device in the bodily cavity. In some example embodiments, the described systems employ a navigation system (e.g., navigation system 342 (also referred to as a catheter navigation system in some embodiments) illustrated in FIGS. 3A and 3B), such as an electro-anatomical mapping system including electromagnetic-based systems or an electropotential-based systems to determine a positioning of a portion of a device in a bodily cavity. U.S. Pat. No. 11,918,303, issued Mar. 5, 2024 (Moisa) describes such navigation systems, and such descriptions are hereby incorporated herein by reference. In some embodiments, the navigation system (e.g., navigation system 342) utilizes three pairs of contact patches (e.g., electrodes) (e.g., pairs of contact patches 342x, 342y, and 342z illustrated in FIGS. 3A and 3B) that are placed externally on the patient in order to detect three-dimensional coordinates of an object in the patient's body (e.g., at least part of the catheter device system 200, 300, 400). For instance, contact patch pair 342x may be placed along the sides of the patient (e.g., under the patient's armpits) in order to form an x-axis for location detection, contact patch pair 342y may have one contact patch placed relatively closer to the patient's head and the other contact patch placed relatively closer to the patient's groin to form a y-axis for location detection, and contact patch pair 342z may have one contact patch placed on the patient's chest and the other contact patch placed on the patient's back to form a z-axis for location detection. Additional axes may be provided in some embodiments. Each contact patch pair may be associated with its own signal frequency compared to the other contact patch pairs, so that positions can be detected along each of the at least three axes associated with the catheter navigation system (e.g., navigation system 342) to provide a detection location in three-dimensional space.

In some example embodiments, the described transducer-based devices are part of a transducer-activation system capable of ablating tissue in a desired pattern within the bodily cavity using various techniques (e.g., via thermal ablation, PFA, etc., according to various embodiments).

In some example embodiments, the devices are capable of sensing various cardiac functions (e.g., electrophysiological activity including intracardiac voltages which form the basis of recorded electrograms according to some embodiments). In some example embodiments, the devices are capable of providing stimulation (e.g., electrical stimulation) to tissue within the bodily cavity. Electrical stimulation may include pacing.

FIG. 2 is a representation of a medical system including a transducer-based device 200 (which may also be referred to as a medical device or a catheter device system) useful in investigating or treating a bodily organ, for example, a heart 202, according to at least one example embodiment. In some embodiments in which the transducer-based device 200 is configured at least to transmit pulsed field ablation energy, the transducer-based device 200 may be considered at least part of a pulsed field ablation system.

Transducer-based device 200 can be percutaneously or intravascularly inserted into a portion of the heart 202, such as an intracardiac cavity like left atrium 204. In this example, the transducer-based device 200 is part of a catheter 206 inserted via the inferior vena cava 208 and penetrating through a bodily opening in transatrial septum 210 from right atrium 212. (In this regard, transducer-based devices or device systems described herein that include a catheter may also be referred to as catheter devices, catheter-based devices, catheter device systems, or catheter-based device systems, according to various embodiments). In other embodiments, other paths may be taken.

Catheter 206 includes an elongated flexible rod or shaft member appropriately sized to be delivered percutaneously or intravascularly. Various portions of catheter 206 may be steerable. Catheter 206 may include one or more lumens. The lumen(s) may carry one or more communications or power paths, or both. For example, the lumens(s) may carry one or more electrical conductors 216 (two shown). Electrical conductors 216 provide electrical connections to transducer-based device 200 that are accessible externally from a patient in which the transducer-based device 200 is inserted.

Transducer-based device 200 includes a frame or structure 218 which assumes an unexpanded configuration for delivery to left atrium 204. Structure 218 is expanded (e.g., shown in a deployed or expanded configuration in FIG. 2) upon delivery to left atrium 204 to position a plurality of transducers 220 (three called out in FIG. 2) proximate the interior surface formed by tissue 222 of left atrium 204. In some embodiments, at least some of the transducers 220 are used to sense a physical characteristic of a fluid (e.g., blood) or tissue 222, or both, that may be used to determine a position or orientation (e.g., pose), or both, of a portion of a device 200 within, or with respect to left atrium 204. For example, transducers 220 may be used to determine a location of pulmonary vein ostia or a mitral valve 226, or both. In some embodiments, at least some of the transducers 220 may be used to selectively ablate portions of the tissue 222. For example, some of the transducers 220 may be used to ablate a pattern around the bodily openings, ports or pulmonary vein ostia, for instance to reduce or eliminate the occurrence of atrial fibrillation. In some embodiments, at least some of the transducers 220 are used to ablate cardiac tissue. In some embodiments, at least some of the transducers 220 are used to sense or sample intracardiac voltage data or sense or sample intracardiac electrogram data. In some embodiments, at least some of the transducers 220 are used to sense or sample intracardiac voltage data or sense or sample intracardiac electrogram data while at least some of the transducers 220 are concurrently ablating cardiac tissue. In some embodiments, at least one of the sensing or sampling transducers 220 is the same as at least one of the ablating transducers 220. In at least some embodiments at least in which at least one of the sensing or sampling transducers 220 is the same as at least one of the ablating transducers 220, each of the at least one transducer 220 may operate in multiple modes including a first mode in which it is configured as a transducer configured to deliver tissue ablation energy and a second mode in which it is configured as a sensor (e.g., electrophysiological activity sensor). In some embodiments, at least a first one of the transducers 220 senses or samples intracardiac voltage data or intracardiac electrogram data at a location at least proximate to a tissue location ablated by at least a second one of the transducers 220. In some embodiments, the first one of the transducers 220 is other than the second one of the transducers 220.

FIGS. 3A and 3B illustrate a medical system (e.g., a portion thereof shown schematically) that includes a transducer-based device 300, which may also be referred to as a catheter device system or an electrode-based device, according to various embodiments. In some embodiments in which the transducer-based device 300 is configured at least to transmit pulsed field ablation energy, the transducer-based device 300 may be considered at least part of a pulsed field ablation system.

All or part of such catheter device system may be all, or part of, a tissue ablation system or sampling or sensing system, according to various embodiments. All or part of such catheter device system may be all or part of a medical system, such as system 100 shown in FIG. 1, according to various embodiments. As noted above, the controller 324 may be a particular implementation of the data processing device system 110 shown in FIG. 1.

The catheter device system 300 (or transducer-based device, or electrode-based device) may be the same as or similar to the catheter device system 200, although different sizes, numbers of transducers, or types of medical devices, such as balloon catheters, may be implemented. In this regard, catheter device system 300 includes a plurality of elongate members 304 (not all of the elongate members are called out in FIGS. 3A and 3B) and a plurality of transducers 306 (not all of the transducers are called out in FIGS. 3A and 3B; some of the transducers 306 are called out in FIG. 3B as 306a, 306b, and 306c). FIG. 3A includes a representation of a portion of the catheter device system 300 in a delivery or unexpanded configuration. FIG. 3B includes a representation of a portion of the catheter device system 300 in an expanded or deployed configuration. It is noted that, for clarity of illustration, all of the elongate members shown in FIG. 3B are not represented in FIG. 3A. As will become apparent, the plurality of transducers 306 is positionable within a bodily cavity, such as with the catheter device system 200. For example, in some embodiments, the transducers 306 may be positioned in a bodily cavity by movement of at least part of the elongate members 304 into, within, or into and within the bodily cavity, with or without a change in a configuration of the plurality of transducers 306. In some embodiments, the transducers of the plurality of transducers 306 are arranged to form a two- or three-dimensional distribution, grid or array of the transducers capable of mapping, ablating, or stimulating an inside surface of a bodily cavity or lumen without requiring mechanical scanning. As shown, for example, in FIG. 3A, the plurality of transducers 306 are arranged in a distribution receivable in a bodily cavity. In FIGS. 3A and 3B, each of at least some of transducers 306 includes a respective electrode 315 (not all of the electrodes 315 are called out in FIGS. 3A and 3B).

The elongate members 304 are arranged in a frame or structure 308 that is selectively moveable between an unexpanded or delivery configuration (e.g., as shown in FIG. 3A) and an expanded or deployed configuration (e.g., as shown in at least FIG. 3B) that may be configured to position elongate members 304 against a tissue surface within the bodily cavity or position the elongate members 304 in the vicinity of the tissue surface. In some embodiments, structure 308 has a size in the unexpanded or delivery configuration suitable for delivery through a bodily opening (e.g., via catheter sheath 312) to the bodily cavity. In various embodiments, catheter sheath 312 typically includes a length sufficient to allow the catheter sheath to extend between a location at least proximate a bodily cavity into which the structure 308 is to be delivered and a location outside a body comprising the bodily cavity. In some embodiments, structure 308 has a size in the expanded or deployed configuration too large for delivery through a bodily opening (e.g., via catheter sheath 312) to the bodily cavity. The elongate members 304 may form part of a flexible circuit structure (e.g., also known as a flexible printed circuit board (PCB) circuit, examples of which are described with respect to FIG. 4, below). The elongate members 304 may include a plurality of different material layers. Each of the elongate members 304 may include a plurality of different material layers. The structure 308 may include a shape memory material, for instance, Nitinol. The structure 308 may include a metallic material, for instance, stainless steel, or non-metallic material, for instance, polyimide, or both a metallic and non-metallic material by way of non-limiting example. The incorporation of a specific material into structure 308 may be motivated by various factors including the specific requirements of each of the unexpanded or delivery configuration and expanded or deployed configuration, the required position or orientation (e.g., pose), or both of structure 308 in the bodily cavity, the requirements for successful ablation of a desired pattern, or the effect that the material may have on navigation electric or magnetic fields to be sensed by the device (e.g., by one or more sensors configured to sense a voltage caused or induced by at least one electric field or magnetic field respectively).

FIG. 4 is a schematic side elevation view of at least a portion of a transducer-based device (or electrode-based device) 400 that includes a flexible circuit structure 401 that is employed to provide a plurality of transducers 406 (two called out) according to an example embodiment. In some embodiments, the flexible circuit structure 401 may form part of a structure (e.g., structure 308) that is selectively moveable between a delivery configuration sized for percutaneous delivery and an expanded or deployed configuration sized too large for percutaneous delivery. In some embodiments, the flexible circuit structure 401 may be located on, or form at least part of, a structural component (e.g., elongate member 304) of a transducer-based device system.

The flexible circuit structure 401 can be formed by various techniques including flexible printed circuit techniques. In some embodiments, the flexible circuit structure 401 includes various layers including flexible layers 403a, 403b and 403c (e.g., collectively flexible layers 403). In some embodiments, each of flexible layers 403 includes an electrical insulator material (e.g., polyimide). One or more of the flexible layers 403 can include a different material than another of the flexible layers 403. In some embodiments, the flexible circuit structure 401 includes various electrically conductive layers 404a, 404b, and 404c (collectively electrically conductive layers 404) that are interleaved with the flexible layers 403. In some embodiments, each of the electrically conductive layers 404 is patterned to form various electrically conductive elements. For example, electrically conductive layer 404a is patterned to form a respective electrode 415 of each of the transducers 406. Electrodes 415 have respective electrode edges 415-1 that form a periphery of an electrically conductive surface associated with the respective electrode 415. It is noted that other electrodes employed in other embodiments may have electrode edges arranged to form different electrodes shapes (for example, as shown by electrode edges 315-1 in FIG. 3B).

Electrically conductive layer 404b is patterned, in some embodiments, to form respective temperature sensors 408 for each of the transducers 406 as well as various leads 410a arranged to provide electrical energy to the temperature sensors 408. In some embodiments, each temperature sensor 408 includes a patterned resistive member 409 (two called out, each an example of an RTD) having a predetermined electrical resistance. In some embodiments, each resistive member 409 includes a metal having relatively high electrical conductivity characteristics (e.g., copper). In various embodiments, a thermistor or a thermocouple may be implemented. In some embodiments, electrically conductive layer 404c is patterned to provide portions of various leads 410b arranged to provide an electrical communication path to electrodes 415. In some embodiments, leads 410b are arranged to pass through vias in flexible layers 403a and 403b to connect with electrodes 415. Although FIG. 4 shows flexible layer 403c as being a bottom-most layer, some embodiments may include one or more additional layers underneath flexible layer 403c, such as one or more structural layers, such as a steel or composite layer. These one or more structural layers, in some embodiments, are part of the flexible circuit structure 401 and can be part of, e.g., elongate member 304. In some embodiments, the one or more structural layers may include at least one electrically conductive surface (e.g., a metallic surface) exposed to blood flow. In addition, although FIG. 4 shows only three flexible layers 403a-403c and only three electrically conductive layers 404a-404c, it should be noted that other numbers of flexible layers, other numbers of electrically conductive layers, or both, can be included.

In some embodiments, electrodes 415 are employed to selectively deliver ablation energy (e.g., thermal ablation energy or PFA energy) to various tissue structures within a bodily cavity (e.g., an intracardiac cavity or chamber). The energy delivered to the tissue structures may be sufficient for ablating portions of the tissue structures. The energy delivered to the tissue may be delivered to cause monopolar tissue ablation, bipolar tissue ablation or blended monopolar-bipolar tissue ablation by way of non-limiting example.

Energy that is sufficient for tissue ablation may be dependent upon factors including transducer location, size, shape, relationship with respect to another transducer or a bodily cavity, material or lack thereof between transducers, et cetera. For example, in “RF” ablation, a larger electrode (e.g., an electrode with a relatively large surface area) will achieve a given ablation depth sooner than a smaller electrode. Stated differently, a maximum ablation depth of a relatively smaller electrode is typically shallower than that of a relatively larger electrode when ablating under the same control parameters as a relatively larger electrode.

In some embodiments, each electrode 415 may be configured, at least in a particular mode of operation, to sense or sample electrophysiological activity information. In some embodiments, each electrode 415 may be configured, at least in a particular mode of operation, to sense or sample an electrical potential in the tissue proximate the electrode 415 (e.g., at a different time than delivering energy sufficient for tissue ablation, in some embodiments). In some embodiments, each electrode 415 may be configured, at least in a particular mode of operation, to sense or sample intracardiac voltage data in the tissue proximate the electrode 415. In some embodiments, each electrode 415 may be configured, at least in a particular mode of operation, to sense or sample data (e.g., indicating electrophysiological activity) in the tissue proximate the electrode 415 from which an electrogram may be derived. In some embodiments, each resistive member 409 is positioned adjacent a respective one of the electrodes 415. In some embodiments, each of the resistive members 409 is positioned in a stacked or layered array with a respective one of the electrodes 415 to form a respective one of the transducers 406. In some embodiments, the resistive members 409 are connected in series to allow electrical current to pass through all of the resistive members 409. In some embodiments, leads 410a are arranged to allow for a sampling of electrical voltage in between resistive members 409. This arrangement allows for the electrical resistance of each resistive member 409 to be accurately measured. The ability to accurately measure the electrical resistance of each resistive member 409 may be motivated by various reasons including determining temperature values at locations at least proximate the resistive member 409 based at least on changes in the resistance caused by convective cooling effects (e.g., as provided by blood flow).

Referring to FIGS. 3A and 3B, catheter device system 300 is configured to communicate with, receive power from, or be controlled by a transducer-activation device system 322 according to some embodiments. In some embodiments, at least part of the transducer-activation device system 322 represents one or more particular implementations of the system 100 illustrated in FIG. 1. In some embodiments, elongate members 304 include a plurality of transducers 306 (and electrodes 315 thereof) that are communicatively connectable or connected to an input-output device system 320 of a data processing device system 310 via electrical connections running within elongate shaft member 314 that are communicatively connected to one or more of electrical leads 317 (e.g., control leads, data leads, power leads or any combination thereof) within elongated cable 316 (only a portion of which is shown in FIGS. 3A and 3B to reveal other structures) terminating at a connector 321 or other interface. The connector 321 may be a removable-type connector, like a plug, in some embodiments, to connect to and disconnect from the controller 324, according to some embodiments. The transducer-activation device system 322 may include a controller 324 that includes the data processing device system 310 (e.g., which may be a particular implementation of data processing device system 110 from FIG. 1) and a memory device system 330 (e.g., which may be a particular implementation of the memory device system 130 from FIG. 1) that stores data and instructions that are executable by the data processing device system 310 to process information received from catheter device system 300 or to control operation of catheter device system 300, for example, activating various selected transducers 306 to ablate tissue and control a user interface (e.g., of input-output device system 320) according to various embodiments. Controller 324 may include one or more controllers.

Transducer-activation device system 322 includes an input-output device system 320 (e.g., which may be a particular implementation of the input-output device system 120 from FIG. 1) communicatively connected to the data processing device system 310 (e.g., via controller 324 in some embodiments). Input-output device system 320 may include a user-activatable control that is responsive to a user action. Input-output device system 320 may include one or more user interfaces or input/output (I/O) devices, for example, one or more display device systems 332, speaker device systems 334, one or more keyboards, one or more mice (e.g., mouse 335), one or more joysticks, one or more track pads, one or more touch screens or other transducers to transfer information to, from, or both to and from a user, for example a care provider such as a physician or technician. For example, output from a mapping process may be displayed by a display device system 332. Input-output device system 320 may include one or more user interfaces or input/output (I/O) devices, for example, one or more display device systems 332, speaker device systems 334, keyboards, mice, joysticks, track pads, touch screens or other transducers employed by a user to indicate a particular selection or series of selections of various graphical information. Input-output device system 320 may include a sampling or sensing device system 325 configured to detect various characteristics including, but not limited to, at least one of tissue characteristics (e.g., electrical characteristics such as tissue impedance, electric potential of a tissue surface, tissue conductivity, tissue type, tissue thickness) and thermal characteristics such as temperature. In this regard, the sampling or sensing device system 325 may include one, some, or all, of the transducers 306 (or 220 in FIG. 2 or 406 of FIG. 4) of the catheter device system 300, including the internal components of such transducers shown in FIG. 4, such as the electrodes 415 and temperature sensors 408 or other temperature sensors. Input-output device system 320 may also include navigation system 342 (described above), which may provide navigation information to the controller 324 or data processing device system 110, 310 indicating, e.g., in three-dimensional space, a position of at least part of the catheter device system 300 (or catheter device system 200 (or 400 discussed above)) within the bodily cavity as well as an orientation of the bodily cavity from which various patient axes (discussed in more detail below) may be determined by the controller 324 or data processing device system 110, 310, according to various embodiments.

Transducer-activation device system 322 may also include an energy source device system 340 including one or more energy source devices connected to transducers 306. In this regard, although FIGS. 3A and 3B show a communicative connection between the energy source device system 340 and the controller 324 (and its data processing device system 310), the energy source device system 340 may also be connected to the transducers 306 via a communicative connection that is independent of the communicative connection with the controller 324 (and its data processing device system 310). For example, the energy source device system 340 may receive control signals via the communicative connection with the controller 324 (and its data processing device system 310), and, in response to such control signals, deliver energy to, receive energy from, or both deliver energy to and receive energy from one or more of the transducers 306 via a communicative connection with such transducers 306 (e.g., via one or more communication lines through the catheter body or elongate shaft member 314, elongated cable 316 or catheter sheath 312 to such transducers 306) that does not pass through the controller 324. The energy source device system 340 may provide results of its delivering energy to, receiving energy from, or both delivering energy to and receiving energy from one or more of the transducers 306 to the controller 324 (and its data processing device system 310) via the communicative connection between the energy source device system 340 and the controller 324.

The energy source device system 340 may, for example, be connected to various selected transducers 306 to selectively provide energy in the form of electrical current or power, light, low-temperature fluid, or another form to the various selected transducers 306 to cause ablation of tissue. The energy source device system 340 may, for example, selectively provide energy in the form of electrical current to various selected transducers 306 and measure a temperature characteristic, an electrical characteristic, or both at a respective location at least proximate each of the various transducers 306. The energy source device system 340 may include various electrical current sources or electrical power sources as energy source devices. In some embodiments, an indifferent electrode 326 is provided to receive at least a portion of the energy transmitted by at least some of the transducers 306. Consequently, although not shown in FIGS. 3A and 3B, the indifferent electrode 326 may be communicatively connected to the energy source device system 340 via one or more communication lines in some embodiments. In addition, although shown separately in each of FIGS. 3A and 3B, indifferent electrode 326 may be considered part of the energy source device system 340 in some embodiments. In various embodiments, indifferent electrode 326 is positioned on an external surface (e.g., a skin-based surface) of a body that comprises the bodily cavity into which at least transducers 306 are to be delivered.

It is understood that input-output device system 320 may include other systems. In some embodiments, input-output device system 320 may optionally include energy source device system 340, catheter device system 300 or both energy source device system 340 and catheter device system 300 by way of non-limiting example. Input-output device system 320 may include the memory device system 330 in some embodiments.

In other example embodiments, other structures besides those shown in FIGS. 2, 3A, 3B, and 4 may be employed to support or carry transducers of a transducer-based device such as a transducer-based catheter. For example, an elongated catheter member may be used to distribute the transducers in a linear or curvilinear array. Basket catheters or balloon catheters may be used to distribute the transducers in a two-dimensional or three-dimensional array.

According to various embodiments, the plurality of transducers of the catheter device system (e.g., catheter device system 200, 300, 400) are positionable within a bodily cavity of a patient. For example, the catheter device system may include a structure (e.g., structure 218 or 308) that is configured to support various transducers and to be deliverable to, or positionable within, a bodily cavity, thereby, e.g., being configured to place such transducers within the bodily cavity. According to some embodiments, each transducer of at least some transducers of the plurality of transducers or each transducer of the plurality of transducers (e.g., transducers 220, 306, 406) is configured to ablate tissue and sense the electrophysiological activity information. For example, in some embodiments, each transducer (or electrode) of the at least some transducers or each transducer (or electrode) of the plurality of transducers (e.g., transducers 220, 306, 406) is configured to transmit particular energy configured to cause pulsed field ablation of tissue. In some embodiments, each transducer of the at least some transducers or each transducer of the plurality of transducers is configured to transmit particular energy configured to cause pulsed field ablation of tissue. In some embodiments, each transducer of the at least some transducers or each transducer of the plurality of transducers may be configured to record a respective electrogram. For example, in some embodiments, each transducer of the at least some transducers or each transducer of the plurality of transducers may include an electrode (e.g., 315, 415), each electrode configured to (a) transmit tissue ablative energy, (b) record electrograms, or (a) and (b), according to various embodiments. In some embodiments, each transducer of the at least some transducers or each transducer of the plurality of transducers may include a particular transducer or particular electrode (e.g., 315, 415), that may be configured to provide, in conjunction with a navigation or tracking system, location or position information.

According to various embodiments, the transducers of the plurality of transducers (e.g., transducers 220, 306, 406) may be individually selectable. According to various embodiments, various transducer sets of the plurality of transducers (e.g., transducers 220, 306, 406) may be individually selectable. According to various embodiments, the transducers of the plurality of transducers (e.g., transducers 220, 306, 406) may be individually addressable for identification for selection, ablation, or sensing. According to various embodiments, different parts of at least a portion of the catheter device system may be manipulable to, in turn, manipulate various ones of the plurality of transducers (e.g., transducers 220, 306, 406) into various degrees of contact with a tissue wall within a patient's body (e.g., within a bodily cavity within the patient's body).

According to various embodiments, each transducer of at least some transducers of the plurality of transducers (e.g., transducers 220, 306, 406) may be configured at least to sense a degree of contact between the transducer and the tissue wall. In some embodiments, each particular transducer of at least some transducers of the plurality of transducers may be configured to sense or detect a degree or amount of transducer-to-tissue contact between at least a portion of the particular transducer and the tissue wall. Various methods may be executed to determine the degree or amount of transducer-to-tissue contact including, by way of non-limiting example, techniques including sensing or sampling impedance, sensing or sampling permittivity, sensing or sampling the presence or absence of flow of a fluid (e.g., a bodily fluid), or by sensing or sampling contact force or pressure. U.S. Pat. No. 8,906,011, issued Dec. 9, 2014 (Gelbart et al.), describes example transducer sensing techniques to sense tissue contact. In some embodiments, the tissue-contacting portion of the transducer itself directly senses or samples the degree of tissue contact. In some embodiments, a portion of the transducer other than the tissue-contacting portion of the transducer is configured to sense or sample the degree of contact between the tissue wall and the tissue-contacting portion of the transducer. In some embodiments, the tissue-contacting portion of the transducer is provided by an electrode. According to some embodiments, at least some transducers of the plurality of transducers of the catheter device system (e.g., 200, 300, 400) may be configured to provide a plurality of contact signal sets to the controller 324 or its data processing device system 310. The plurality of contact signal sets may be referred to as degree of transducer-to-tissue contact information in some embodiments. In this regard, each contact signal set may provide or indicate a degree of transducer-to-tissue contact between each transducer (e.g., a transducer 220, 306, 406) and a tissue surface in the bodily cavity.

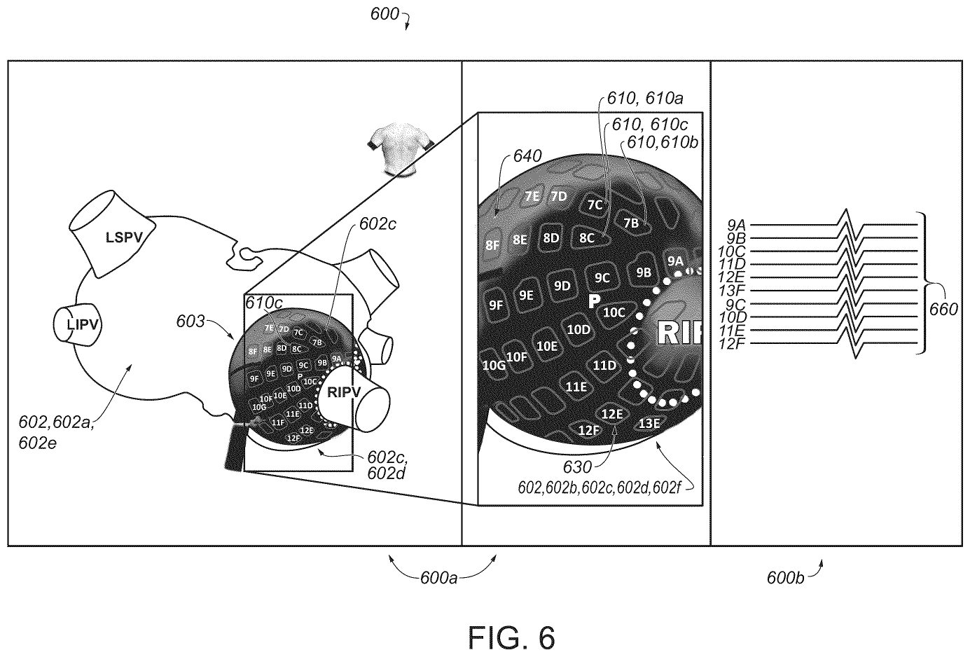

FIG. 6, discussed in more detail below, shows a graphical display including a graphical representation 600 (e.g., displayed via the input-output device system 120, 320) of a map 602 showing a volumetric region 602a within a bodily cavity (which is a heart cavity in this example) and a tissue region 602b exhibiting transducer-to-tissue contact, according to some embodiments. In this regard, the input-output device system 120, 320 may include any suitable interface for outputting information (e.g., transducer-to-tissue contact information), instructions, or data to other devices and systems described in various ones of the embodiments. In this regard, the input-output device system 120, 320 may include various other devices or systems described in various embodiments. In some embodiments, the input-output device system 120, 320 may include one or more display devices that display one or more of the graphical interfaces and graphical representations of FIG. 6.

According to some embodiments, the data processing device system (e.g., 110, 310) may be configured (e.g., by computer-executable instructions, such as display instructions provided by a program) to cause, via the input-output device system (e.g., 120, 320), display of a graphical representation, such as graphical representation 600 in FIG. 6. The graphical representation may include a map, according to some embodiments. In some embodiments, the map may include a plurality of regions. Such regions may include volumetric regions within a body (e.g., based on navigation information from navigation system 342), tissue regions of a tissue surface of a bodily cavity, or both volumetric regions within a bodily cavity and tissue regions of a tissue surface, according to some embodiments.

In some embodiments, the map includes a map of at least part of a catheter device system (e.g., catheter device system 200, 300, 400). In some embodiments, the map includes a transducer map of at least some transducers from the plurality of transducers (e.g., transducers 220, 306, 406) of the catheter device system (e.g., 200, 300, or 400). In some embodiments, the graphical representation (e.g., graphical representation 600) may include a display of a graphical labeling set, such as graphical labels identifying transducers in a transducer map. In some embodiments, the graphical representation may include a display of at least part of an electrogram set (e.g., electrogram set 660).

For instance, FIG. 6 shows, according to some embodiments, an example of a graphical representation 600 (or graphical interface or user interface since it may be configured to receive user input in some embodiments). According to some embodiments, the graphical representation 600 includes a first portion 600a and a second portion 600b. The first portion 600a of the graphical representation 600 displays a map, e.g., of a bodily cavity, and the second portion 600b of the displayed graphical representation 600 displays, among other things, a plurality of electrograms. It is noted that, although the first portion 600a and the second portion 600b are shown as forming the entirety of displayed graphical representation 600 in FIG. 6, such need not be the case in other embodiments where, e.g., the first and second portions 600a, 600b form parts of different graphical representations or include additional features. It also is noted that, although the first portion 600a of the graphical representation 600 is shown to the left of the second portion 600b of the graphical representation, other arrangements may be used in various embodiments. The graphical representation 600, or different portions thereof, may be displayed via multiple display devices in some embodiments.

The first portion 600a of the displayed graphical representation 600 may be used to visually display a map 602 of at least part of a bodily cavity. In FIG. 6, the map 602 is displayed in both a zoomed-out view 602e and a zoomed-in view 602f, according to some embodiments. In the example of FIG. 6, the map 602 includes or is of at least part of an atrium of a heart, where “RIPV” shown in the map 602 means “right inferior pulmonary vein”, “LSPV” means “left superior pulmonary vein”, and “LIPV” means “left inferior pulmonary vein”. In some embodiments, the map 602 is generated (e.g., by the data processing device system 110, 310) at least in part from navigation information provided by a navigation system (e.g., an electropotential navigation system or an electromagnetic navigation system, such as navigation system 342 discussed above in this disclosure).

In some embodiments, the display of the map 602 includes or is a display of a geometric surface 602c of a structure (e.g., structure 218 or 308) of the catheter device system (e.g., catheter device system 200, 300, or 400). In some embodiments, the display of the map 602 includes or is a transducer map 602d of at least some transducers of the plurality of transducers (e.g., transducers 220, 306, 406) of the catheter device system. The transducers may be represented as transducer graphical elements 610 (a few called out in FIG. 6, with one called out with reference 610a, one called out with reference 610b, and one called out with reference 610c), according to some embodiments.

In some embodiments, a list of graphical elements identifying particular ones of the plurality of transducers (e.g., transducers 220, 306, 406) may be displayed. In some embodiments, the plurality of transducers may be arranged according to a first spatial distribution, and the first portion 600a of the graphical representation 600 may include a set of transducer graphical elements 610, where each transducer graphical element 610 graphically represents a respective transducer of the plurality of transducers, and the transducer graphical elements 610 may be graphically arranged according to a second spatial distribution that is consistent with the first spatial distribution. Such second spatial distribution of the transducer graphical elements 610 may be considered a map of the transducer graphical elements 610, such that, e.g., the graphical representation 600 may be considered to include a graphical display of a transducer map, in some embodiments. In some embodiments, the arrangement (or map) of the transducer graphical elements 610 is graphically represented in a three-dimensional manner (for example, as shown in FIG. 6). In FIG. 6, a three-dimensional graphical representation 603 including a three-dimensionally represented arrangement of transducer graphical elements 610 corresponds to at least a portion of the catheter device system or transducer-based device that is similar to the spherical or quasi-spherical arrays of transducers shown in FIGS. 2 and 3B. In some embodiments, the arrangement (or map) of the transducer graphical elements 610 is graphically represented in a two-dimensional manner. In some embodiments, the transducer graphical elements 610 in FIG. 6 may correspond to transducers 220, 306, 406.

In some embodiments, each transducer graphical element 610 may be identified by a graphical label including a numerical identifier and an alphabetical identifier, where the numerical identifier indicates along which elongate member portion (e.g., a portion of an elongate member 304) the transducer (e.g., respective transducer 220, 306, 406) associated with that transducer graphical element 610 is located, and the alphabetical identifier indicates where or how far along that elongate member portion the transducer (e.g., respective transducer 220, 306, 406) associated with that transducer graphical element 610 is located. For instance, transducer graphical element 610b in FIG. 6 is graphically labeled by number “7” and letter “B”, where the number “7” indicates that the transducer graphical element 610b is associated with a corresponding transducer (e.g., transducer 220, 306, 406) that is located along what is considered the seventh elongate member portion at position “B” along that elongate member portion. Of course, other manners of uniquely identifying a transducer besides a number and letter designation may be used in various embodiments.

In some particular embodiments in which the at least the portion of the catheter device system (e.g., catheter device system 200, 300, 400) includes a plurality of transducers (e.g., transducers 220, 306, 406), the graphical representation (e.g., 600) maps information indicating a spatial distribution of transducer-to-tissue contact information. For example, in FIG. 6, the darker-shaded region 630 indicates greater degrees of transducer-to-tissue contact than the more lightly shaded region 640, which indicates lesser degrees of or no transducer-to-tissue contact. Although FIG. 6 utilizes grayscale shading to differentiate between high-contact region 630 and low-contact region 640, it should be noted that other visual indications of degrees of detected transducer-to-tissue contact may be utilized, according to various embodiments.

According to some embodiments, the data processing device system 110, 310 may be configured by the program at least to cause display of the graphical representation (e.g., graphical representation 600) to include at least part of an electrogram set. In this regard, in some embodiments in which the sensing or sampling, e.g., discussed in more detail below with respect to at least FIG. 5, is of electrophysiological activity (or electrophysiological activity information), the data processing device system (e.g., 110, 310) configured at least by the program at least to cause, via the input-output device system (e.g., 120, 320), display of a plurality of electrograms (e.g., electrogram set 660 shown in FIG. 6) derived from the plurality of sensings or samplings of the electrophysiological activity (or electrophysiological activity information). For instance, the second portion 600b of the graphical representation 600 in FIG. 6 displays a plurality of electrograms as at least part of electrogram set 660, according to some embodiments. It should be noted that the electrograms shown in FIG. 6 are merely simplified illustrative electrograms for purposes of clarity, and actual electrograms would likely differ in appearance. Further, although the electrograms shown in FIG. 6 show generally the same propagation (activation) timings of electrogram signals for ease of illustration, it is likely that the electrograms would exhibit slight offsets in propagation (activation) timings due to the different locations associated with respective transducers that detect the electrograms. In some embodiments, the display of the electrogram set 660 is a visual stacking or ordering of electrograms, e.g., as shown at least in FIG. 6. In some embodiments, the displayed electrogram set 660 may not show all electrograms recorded by the plurality of transducers (e.g., transducers 220, 306, 406), particularly in embodiments where there are a greater number of transducers than electrograms that can reasonably be displayed in the graphical representation 600. For instance, in FIG. 6, the displayed electrogram set 660 shows ten (10) electrograms (e.g., by way of non-limiting example), although there may be a much greater number of electrograms recorded by the plurality of transducers represented by transducer graphical elements 610.

According to various embodiments, the electrogram set 660 is produced by electrophysiological activity information, such as electrophysiological voltage data, recorded internally within the body. The electrogram set 660 may be recorded by at least some of the transducers of the plurality of transducers (e.g., transducers 220, 306, 406), according to some embodiments. The electrograms may include unipolar electrograms, bipolar electrograms, or both unipolar electrograms and bipolar electrograms. In some embodiments, the electrogram may include omnipolar electrograms.

In FIG. 6, according to some embodiments, each electrogram of the electrogram set 660 corresponds to, or is recorded by, a respective transducer or transducer set of the plurality of transducers. For instance, an electrode of a respective transducer of the plurality of transducers 220, 306, 406 may record or at least participate in recording (e.g., in a bipolar case with another electrode) an electrogram of the electrogram set 660, where the electrode is configured as a sensor in some embodiments to sense or sample electrophysiological voltages, e.g., via the electrical conductivity of the electrode itself and without utilizing supplied power (e.g., from a system power supply) to enable the sensing of the electrophysiological voltages. Accordingly, in some embodiments, each electrogram is identified in the graphical representation 600 with at least one instance of a combination of a number and a letter to identify a respective one of the transducers and the respective transducer graphical element 610 (for example, as described above in this disclosure) associated with the respective electrogram. According to some embodiments associated with FIG. 6, the electrograms in electrogram set 660 are presented consecutively in an ordered array based on some of the viewable transducer graphical elements 610 visible in the state of the graphical representation shown in FIG. 6, starting with the electrogram recorded by the transducer “9A”, followed by the electrogram recorded by the transducer “9B”, followed by the electrogram recorded by the transducer “10C”, followed by the electrogram recorded by the transducer “11D”, followed by the electrogram recorded by the transducer “12E”, followed by the electrogram recorded by the transducer “13F”, followed by the electrogram recorded by the transducer “9C”, followed by the electrogram recorded by the transducer “10D”, followed by the electrogram recorded by the transducer “11E”, followed by the electrogram recorded by the transducer “12F”, in this example. Of course, other embodiments may display others of the electrograms recorded by various transducers (e.g., transducers 220, 306, 406) and may display them in other sequences, orientations, arrangements, or as other types, such as bipolar electrograms according to some embodiments.

According to various embodiments, other electrograms may not be visually displayed since they number too many to be included in the visually displayed portion 600b of graphical representation 600. It is noted that, according to various embodiments, some of the electrograms that are not visually displayed may, based on user input, for example, be visually displayed. For example, the ordered array of electrogram set 660 may be manipulated (e.g., scrolled by pressing of a directional keyboard key or rotation of a mouse wheel or a directional sliding of a finger on a touch screen, etc.) to visually display electrograms that were not displayed. It is noted that at least some of the electrograms that were visually displayed prior to such manipulation may not be visually displayed just after such manipulation.

In some embodiments, the example graphical representation 600 shown in FIG. 6 may also be configured as a user interface that may be configured to provide for and indicate selection of one or more transducers (e.g., and the associated one or more electrodes) to, for example, deliver pulsed field ablation energy, sense or sample electrophysiological activity, sense or sample other bodily cavity parameters such as temperature or blood flow, or a combination thereof, according to various embodiments. FIG. 5 includes respective data generation and flow diagrams, which may implement various embodiments of methods 500 by way of associated computer-executable instructions according to some example embodiments. In various example embodiments, a memory device system (e.g., memory device systems 130, 330) is communicatively connected to a data processing device system (e.g., data processing device systems 110 or 310, otherwise stated herein as “e.g., 110, 310”) and stores a program executable by the data processing device system to cause the data processing device system to execute various embodiments of methods 500 via interaction with at least, for example, a transducer-based device (e.g., transducer-based device 200, 300, or 400, in some embodiments). In these various embodiments, the program may include instructions configured to perform, or cause to be performed, various embodiments of methods 500.

In some embodiments, the methods 500 may include a subset of the associated blocks or may include additional blocks than those shown in FIG. 5. In some embodiments, the methods 500 may include a different sequence than those indicated between various ones of the associated blocks shown in FIG. 5. The features recited by any block are not intended to be exhaustive or exclusive, and the adopting of any recited features of any particular block in a particular embodiment does not prevent the inclusion of any other features, according to some embodiments, unless the features cannot work together (i.e., are mutually exclusive).

According to some embodiments, methods 500 may include block 502 associated with computer-executable instructions (e.g., first access instructions provided by a program) configured to cause a data processing device system (e.g., 110, 310) to access, via an input-output device system (e.g., 120, 320), first electrogram information indicating a first electrogram set. In some embodiments, the data processing device system is configured by the program at least to receive the first electrogram information from a first electrode set (e.g., one or more electrodes of a respective one or more of the transducers (e.g., 220, 306, 406)).

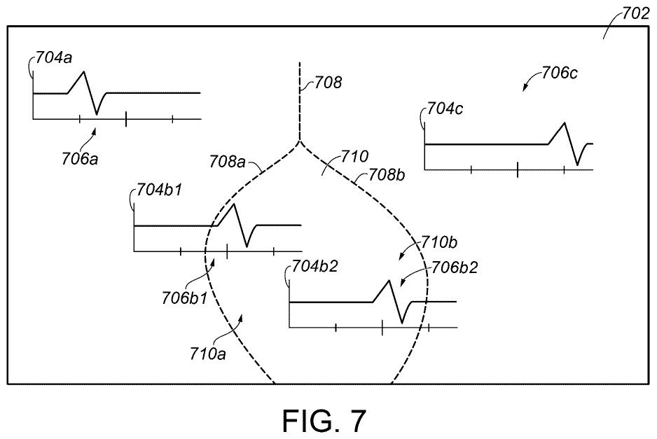

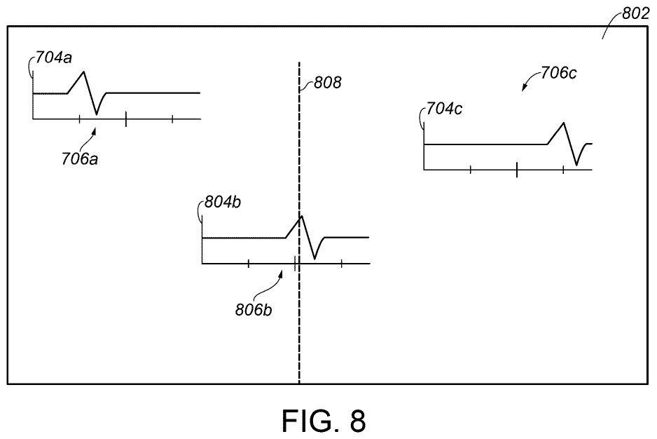

FIG. 7 provides an example in this regard. FIG. 7 illustrates a portion of a map 702 of a bodily cavity (such as a heart cavity in this example) with overlaid electrograms 704a, 704b1, 704b2, and 704c associated with respective locations 706a, 706b1, 706b2, and 706c (e.g., which may be considered or referred to as respective regions in some embodiments) on the portion of the map 702. The overlaid electrograms may or may not be included as part of the map 702 (or map 802 described below with respect to FIG. 8) itself, according to various embodiments. In this regard, in at least some embodiments in which the overlaid electrograms are not included as part of the map 702 (or map 802 described below), such overlaid electrograms may be considered to be shown in FIGS. 7 and 8 in the overlaid manner merely to illustrate to the reader the various features of the various embodiments correspondingly described herein, e.g., without being graphically presented via a display device in the manner shown in FIGS. 7 and 8, according to some embodiments.

In any event, the portion of the map 702 may correspond to a portion of the map 602 shown in FIG. 6, but with overlaid electrograms, e.g., from electrogram set 660, associated with particular locations on the portion of the map 702 corresponding to respective locations where the electrograms were recorded or derived from where the electrograms were recorded. For instance, electrogram 704a (e.g., in the case of a unipolar electrogram) may have been recorded by transducer “9A” (e.g., per FIG. 6) at a first time when it was at a location indicated by location 706a, electrogram 704b1 may have been recorded by transducer “9B” (e.g., per FIG. 6, such transducer “9B” providing an example of the above-mentioned first transducer set, in some embodiments) at the first time when it was at a location indicated by location 706b1, electrogram 704b2 (e.g., in the case of a unipolar electrogram) may have been recorded by transducer “9A” at a second time after the catheter (e.g., catheter device system 200, 300, 400) moved and was then at a location indicated by location 706b2, and electrogram 704c (e.g., in the case of a unipolar electrogram) may have been recorded by transducer “9B” at the second time when it was at a location indicated by location 706c. In the case of a bipolar or omnipolar electrogram that is recorded by multiple transducers, the location represented on or with respect to the map (e.g., map 702 in the example of FIG. 7) for such electrogram may be derived from or selected from the locations of the corresponding transducers that recorded such electrogram, e.g., by taking a mean or median value of at least two of the locations of the corresponding transducers that recorded such electrogram, or by selecting the location of only one of the corresponding transducers that recorded such electrogram, according to various embodiments.

In some embodiments, the accessing of the first electrogram information indicating the first electrogram set per some embodiments of block 502 may be associated with the data processing device system (e.g., 110, 310) accessing the first electrogram information from the memory device system (e.g., 130, 330). With respect to the example of FIG. 7, the first electrogram information may be associated with or describe the electrogram 704b1, such that the electrogram 704b1 is considered the first electrogram set in one example of block 502, according to some embodiments. However, the first electrogram set may include one or more electrograms in various embodiments. In some embodiments, each electrogram in the first electrogram set is a unipolar electrogram. In some embodiments, each electrogram in the first electrogram set is a bipolar electrogram. In some embodiments, each electrogram in the first electrogram set is an omnipolar electrogram. In some embodiments associated with the example of FIG. 7, the first electrogram information may represent sensed electrophysiological activity and may indicate that it was recorded by the transducer “9B” (which is an example of the above-mentioned first transducer set, in some embodiments). However, the first electrogram information indicating the first electrogram set may be associated with one or more electrograms recorded by any one or more transducers (e.g., transducers 220, 306, 406), according to various embodiments. In some embodiments, the first electrogram information may be transmitted from the one or more transducers that recorded the first electrogram set to the data processing device system (e.g., 110, 310) via the input-output device system (e.g., 120, 320) for storage in the memory device system (e.g., 130, 330), according to some embodiments.

According to some embodiments, methods 500 in FIG. 5 may include block 504 associated with computer-executable instructions (e.g., second access instructions provided by a program) configured to cause a data processing device system (e.g., 110, 310) to access, via an input-output device system (e.g., 120, 320), first position information indicating at least a position associated with at least one electrogram in the first electrogram set.

For instance, continuing with the previous example with respect to FIG. 7, the first position information may indicate location 706b1 as an example of the position associated with at least one electrogram in the first electrogram set. In this regard, in some embodiments, the first position information may be associated with a first portion of the bodily cavity (e.g., the first portion including the ‘real space’ location (e.g., as determined from navigation information from a navigation system (e.g., 342) in some embodiments) associated with location 706b1 in the example of FIG. 7). In some embodiments, a position or location associated with a particular electrogram (e.g., as indicated by the first position information associated with block 504, according to some embodiments, or the second position information associated with block 508 discussed in more detail below, according to some embodiments) may be the particular position or location (e.g., in ‘real space’, e.g., as determined from navigation information from a navigation system (e.g., 342) in some embodiments) in which the particular electrogram was recorded, for example, as occurs in the case of a unipolar electrogram. In some embodiments, a position or location associated with a particular electrogram may be a particular position or location derived (e.g., via data processing device system 110, 310) from (e.g., using navigation information from a navigation system (e.g., 342) indicating) the respective positions or locations in which multiple electrograms were recorded, for example, as occurs in the case of a bipolar electrogram or an omnipolar electrogram. For instance, coordinates of the particular position or location may correspond to a mean or median value of the coordinates of the respective positions or locations in which multiple electrograms were recorded. According to some embodiments, a position or location associated with a particular electrogram is determined, or derived, from position or location information provided by navigation system 342.

The first position information associated with block 504 may be recorded in the memory device system (e.g., 130, 330) in association with the first electrogram information (e.g., per block 502), according to some embodiments. In some embodiments, the data processing device system (e.g., 110, 310) may be configured to communicate with a catheter navigation system (e.g., navigation system 342) to determine one or more three-dimensional location(s) or position(s) (e.g., coordinates in some embodiments) of the transducer(s) that recorded the electrogram(s) in the first electrogram set indicated by the first electrogram information (referred to in block 502). The determined one or more three-dimensional locations may be stored as the first position information in the memory device system (e.g., 130, 330), such that it may be accessed by the data processing device system (e.g., 110, 310) per some embodiments of block 504 in FIG. 5, according to some embodiments. In this regard, in some embodiments, the first position information per some embodiments of block 504 may be or may be derived from first navigation information received by the data processing device system (e.g., 110, 310) via the input-output device system (e.g., 120, 320) from a communicatively connected catheter navigation system (e.g., 342).

Continuing with the example of FIG. 7, by having both the first electrogram information and the first position information per some embodiments of blocks 502 and 504 in FIG. 5, the data processing device system (e.g., 110, 310) is able to identify or determine the electrogram 704b1 and its associated location 706b1 in the portion of the map 702. In some embodiments, the first electrogram information and the first position information may indicate first electrophysiological information recorded by at least part of the catheter device system (e.g., 200, 300, 400, such as one or more transducers thereof (e.g., 220, 306, 406)) at a first position (e.g., associated with at least location 706b1 in this example of FIG. 7) in the bodily cavity. In some embodiments, such as in some cases of a bipolar or omnipolar electrogram recorded by multiple transducers, such first position (e.g., associated with at least location 706b1) may be determined by the data processing device system (e.g., 110, 310) from the multiple locations or positions of such multiple transducers, e.g., as described above, according to various embodiments.

Similar to blocks 502 and 504, methods 500 may include blocks 506 and 508 which pertain to second electrogram information and second position information, respectively. In this regard, methods 500 may include block 506 associated with computer-executable instructions (e.g., third access instructions provided by a program) configured to cause a data processing device system (e.g., 110, 310) to access, via an input-output device system (e.g., 120, 320), second electrogram information indicating a second electrogram set. In some embodiments, the data processing device system is configured by the program at least to receive the second electrogram information from a second electrode set (e.g., one or more electrodes of a respective one or more of the transducers (e.g., 220, 306, 406)). In some embodiments, the second electrode set includes at least one electrode that is not in the first electrode set, which is the electrode set that provided the first electrogram information accessed per block 502, according to some embodiments. In some embodiments, at least one electrode from the first electrode set, the second electrode set, or both the first electrode set and the second electrode set is or are configured to cause pulsed field ablation of tissue.

In some embodiments, the accessing of the second electrogram information indicating the second electrogram set per some embodiments of block 506 may be associated with the data processing device system (e.g., 110, 310) accessing the second electrogram information from the memory device system (e.g., 130, 330). With respect to the example of FIG. 7, the second electrogram information may be associated with or describe the electrogram 704b2, such that the electrogram 704b2 is considered the second electrogram set in one example of block 506, according to some embodiments. However, the second electrogram set may include one or more electrograms in various embodiments. In some embodiments, each electrogram in the second electrogram set is a unipolar electrogram. In some embodiments, each electrogram in the second electrogram set is a bipolar electrogram. In some embodiments, each electrogram in the second electrogram set is an omnipolar electrogram. In some embodiments associated with the example of FIG. 7, the second electrogram information may represent sensed electrophysiological activity and may indicate that it was recorded by the transducer “9A” (which may be an example of the above-mentioned second electrode set, in some embodiments). However, the second electrogram information indicating the second electrogram set may be associated with one or more electrograms recorded by any one or more transducers (e.g., transducers 220, 306, 406), according to various embodiments. In some embodiments, the second electrogram information may be transmitted from the one or more transducers that recorded the second electrogram set to the data processing device system (e.g., 110, 310) via the input-output device system (e.g., 120, 320) for storage in the memory device system (e.g., 130, 330), according to some embodiments.

According to some embodiments, methods 500 in FIG. 5 may include block 508 associated with computer-executable instructions (e.g., fourth access instructions provided by a program) configured to cause a data processing device system (e.g., 110, 310) to access, via an input-output device system (e.g., 120, 320), second position information indicating at least a position associated with at least one electrogram in the second electrogram set. The second position information may be identified or determined as discussed above with respect to the first position information and block 504, in some embodiments, and vice versa, in some embodiments.

For instance, continuing with the previous example with respect to FIG. 7, the second position information may indicate location 706b2 as an example of the position associated with at least one electrogram in the second electrode set, such position different than the position associated with the at least one electrogram in the first electrogram set (e.g., location 706b1 in this example of FIG. 7). In some embodiments, the second position information may be associated with a second portion of the bodily cavity (e.g., the second portion including the real space location associated with location 706b2 in the example of FIG. 7). The second position information may be recorded in the memory device system (e.g., 130, 330) in association with the second electrogram information, according to some embodiments. In some embodiments, the data processing device system (e.g., 110, 310) may be configured to communicate with a catheter navigation system (e.g., navigation system 342) to determine one or more three-dimensional location(s) or position(s) (e.g., coordinates in some embodiments) of the transducer(s)/electrode(s) that recorded the electrogram(s) in the second electrogram set indicated by the second electrogram information (referred to in block 504). The determined one or more three-dimensional locations may be stored as the second position information in the memory device system (e.g., 130, 330), such that it may be accessed by the data processing device system (e.g., 110, 310) per some embodiments of block 508 in FIG. 5, according to some embodiments. In this regard, in some embodiments, the second position information per some embodiments of block 508 may be or may be derived from second navigation information received by the data processing device system (e.g., 110, 310) via the input-output device system (e.g., 120, 320) from a communicatively connected catheter navigation system (e.g., 342).

Continuing with the example of FIG. 7, by having both the second electrogram information and the second position information per some embodiments of blocks 506 and 508 in FIG. 5, the data processing device system (e.g., 110, 310) is able to identify or determine the electrogram 704b2 and its associated location 706b2 in the portion of the map 702. In some embodiments, the second electrogram information and the second position information may indicate second electrophysiological information recorded by at least part of the catheter device system (e.g., 200, 300, 400, such as one or more transducers thereof (e.g., 220, 306, 406)) at a second position (e.g., associated with at least location 706b2 in this example of FIG. 7) in the bodily cavity. In some embodiments, such as in some cases of a bipolar or omnipolar electrogram recorded by multiple transducers, such second position (e.g., associated with at least location 706b2 in the example of FIG. 7) may be determined by the data processing device system (e.g., 110, 310) from the multiple locations or positions of such multiple transducers, e.g., as described above, according to various embodiments. In some embodiments, the indicated second position (e.g., associated with at least location 706b2) is different than the first position (e.g., associated with at least location 706b1) indicated by at least the first position information per some embodiments of block 504.

According to some embodiments, methods 500 in FIG. 5 may include block 510 associated with computer-executable instructions (e.g., altering instructions provided by a program) configured to cause a data processing device system (e.g., 110, 310) to alter, based at least on a comparison of the at least one electrogram in the first electrogram set (e.g., referred to in at least block 502) and the at least one electrogram in the second electrogram set (e.g., referred to in at least block 506), the first position information (e.g., from block 504), the second position information (e.g., from block 508), or both the first position information and the second position information.

In this regard, the first position information accessed per block 504 and the second position information accessed per block 508 may be considered to indicate supposed positions or locations associated with each of one or more or all of each of the first and second electrogram sets, respectively, since, as discussed in more detail below, such first and second position information may reflect distortion in a map of the bodily cavity. In some embodiments, such distortion may be detected or determined based at least on a comparison of electrograms in the first and second electrogram sets per some embodiments of block 510.

In some embodiments, the electrograms that are compared for at least part of the comparison referred to in block 510 are all electrograms produced by the transducers (e.g., 220, 306, 406) (e.g., electrodes thereof) of the catheter device system (e.g., 200, 300, 400). In some embodiments, the electrograms that are compared for at least part of the comparison referred to in block 510 are those electrograms whose recording electrodes exhibited at least a threshold degree of electrode-to-tissue contact or a degree of electrode-to-tissue contact within a determined range (e.g., indicating a normal degree of tissue contact that is not too low nor too high) when the respective electrograms were recorded. In some embodiments, in addition to or in lieu of a degree of electrode-to-tissue contact requirement, the electrograms that are compared for at least part of the comparison referred to in block 510 are those electrograms that are within a threshold or other quantified distance metric or range from one or more particular or respective transducers (e.g., 220, 306, 406) (e.g., electrode thereof) or locations on the mapped tissue surface. In some embodiments of such a case, the electrogram(s) associated with (e.g., closest to) the one or more particular or respective one or more transducers or locations on the mapped tissue surface may be considered, in some embodiments, the “first electrogram set” per blocks 502, 504, and 510 compared for similarity per some embodiments of block 510 with the electrogram set, which may be considered, in some embodiments, the “second electrogram set” per blocks 502, 504, and 510, that is within the threshold or other quantified distance metric or range from the one or more particular or respective transducers or locations on the mapped tissue surface. In this regard, after comparing such electrograms and possibly altering corresponding position information per block 510, the process flow of FIG. 5 may loop back from block 510 via loop 514 to block 502, in order to repeat the sequence of blocks 502, 504, 506, 508, and 510 for a next one or more particular or respective transducers or locations on the mapped tissue surface, until the entire map or some portion thereof has been distortion-corrected. In this regard, the loop 514 in FIG. 5 need not only be utilized in this example, and may be utilized in at least in any embodiment in which multiple instances of map distortion are to be corrected.

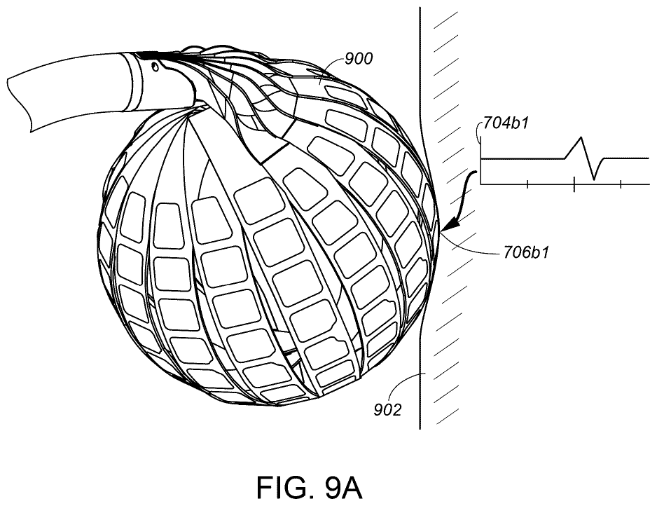

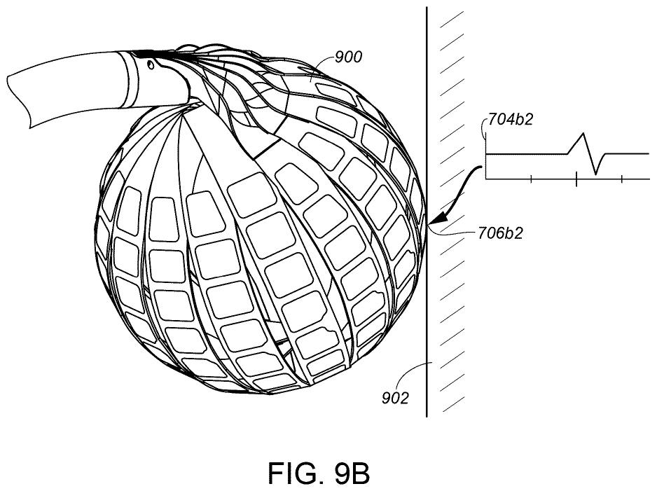

Returning to FIG. 7, a simple example of electrogram comparison per some embodiments of block 510 is illustrated. In this regard, the data processing device system (e.g., 110, 310) may compare the electrogram 704b1 (an example of the first electrogram set per some embodiments of at least block 510) and the electrogram 704b2 (an example of the second electrogram set per some embodiments of at least block 510) and determine that they have a high degree of similarity, indicating that their corresponding originally supposed locations 706b1 and 706b2 may actually represent a same location or closer than originally supposed locations, according to some embodiments. This combination of same or substantially the same electrograms but supposed different locations may indicate a region of distortion in the portion of the map 702, according to some embodiments, as discussed in more detail further below, for example, with respect to FIGS. 9A and 9B. Based on such a comparison, e.g., resulting in a determination of a sufficiently high degree of similarity between the compared electrode sets, the data processing device system (e.g., 110, 310) may be configured per some embodiments of block 510 to alter the originally supposed location 706b1, the originally supposed location 706b2, or both originally supposed locations 706b1 and 706b2 to improve the accuracy of the portion of the map 702. According to various embodiments, a high degree of similarity may indicate a high degree of similarity between the form (e.g., morphology) or one or more parameters of the electrograms, such as voltage amplitudes or timings of the electrograms with respect to a common reference within a cardiac cycle, or a combination of some or all of these items.

For instance, FIG. 8 illustrates an altered portion of the map 702 of the bodily cavity from FIG. 7 resulting in corrected map 802, the altering reducing the distortion of the portion of the map 702, and the altering based at least on a comparison of electrograms 704b1, 704b2, according to various embodiments. In the example improved map state of FIG. 8 compared to the distorted map state of FIG. 7, electrogram 804b may represent a combination of electrograms 704b1 and 704b2 associated with an adjusted location 806b, which, in this example, is between the originally supposed locations 706b1 and 706b2, according to some embodiments.

In some embodiments, if it is determined that the electrogram comparison per some embodiments of block 510 results in a high degree of similarity, the resulting electrogram set (e.g., electrogram 804b) after the position altering may simply use the data (e.g., voltage values over time) of any of the compared electrograms (e.g., electrogram 704b1 or electrogram 704b2) since they are deemed to have a required degree of similarity. In some embodiments, the resulting electrogram set (e.g., electrogram 804b) may be an averaging or median of the compared electrograms (e.g., electrogram 704b1 or electrogram 704b2), according to some embodiments.

In some embodiments, the resulting position set (e.g., location 806b) associated with the resulting electrogram set (e.g., electrogram 804b) may be selected to be one or more of the locations associated with the compared electrograms (a selection of either location 706b1 or location 706b2 in the example of FIG. 7), for instance, in a state in which the data processing device system (e.g., 110, 310) is able to determine that such one or more of the locations is associated with a non-distorted or less-distorted state of the corresponding portion of the map of the bodily cavity, as discussed in more detail below. In some embodiments, the resulting position set (e.g., location 806b) associated with the resulting electrogram set (e.g., electrogram 804b) may be an average or median of the locations of the compared electrograms (e.g., an averaging of locations 706b1 and 706b2). In some embodiments, the resulting position set (e.g., location 806b) associated with the resulting electrogram set (e.g., electrogram 804b) may be a weighted average of the locations of the compared electrograms. For instance, in some embodiments, the data processing device system (e.g., 110, 310) may be configured to further compare each electrogram of the electrograms compared per some embodiments of block 510 with one or more next closest neighbor electrograms (e.g., according to the associated locations) such that the electrogram(s) that is or are more similar to their next closest neighbor(s) are more heavily weighted in the averaging so that the resulting position set (e.g., location 806b) associated with the resulting electrogram set (e.g., electrogram 804b) is located closer to the electrogram(s) from the electrograms compared per some embodiments of block 510 that are more similar to their next closest neighbor(s). For instance, with respect to the example of FIG. 7, if electrogram 704b1 was more similar to its neighbor electrogram 704a than electrogram 704b2 was to its neighbor electrogram 704c, then the resulting position 806b of the resulting electrogram 804b would be closer to the position 706b1 associated with the electrogram 704b1 than it would be to the position 706b2 associated with the electrogram 704b2, according to some embodiments.

In some embodiments, the weighted averaging may reflect a confidence of each electrogram's signal quality or data integrity, where electrograms that are determined to be of greater signal quality or data integrity are weighted more than electrograms determined to be of lesser signal quality or data integrity, such that the resulting position set is skewed more toward electrograms exhibiting greater signal quality or data integrity. For instance, in some embodiments, the resulting position set associated with the resulting electrogram set may be a weighted average of the locations corresponding to the compared electrograms, where the weighted average is dependent on the degrees of electrode-to-tissue contact experienced by the electrodes at the time that they recorded the compared electrograms. For instance, electrograms recorded with a degree of electrode-to-tissue contact determined to be normal may be determined to have greater signal quality or data integrity and, therefore, associated with a heavier weight compared to electrograms recorded with a less-than-normal degree of electrode-to-tissue contact. In some embodiments, an electrode recorded with an excessive, greater-than-normal, degree of electrode-to-tissue contact may be determined to be associated with excessive pressure on the tissue wall of the bodily cavity, causing distortion of the tissue wall as discussed below in greater detail with respect to FIGS. 9A and 9B. In such a case, such electrogram associated with the excessive, greater-than-normal, degree of electrode-to-tissue contact may be associated with a lower weight compared to electrograms recorded with a normal degree of electrode-to-tissue contact. In this regard, other embodiments may use other factors in addition to or in lieu of factors discussed above that impact an electrogram's signal quality or data integrity when performing a weighting function to determine the resulting position set.

FIGS. 9A and 9B illustrate one manner in which map distortion, represented, e.g., by electrograms 704b1 and 704b2 and their corresponding associated locations 706b1 and 706b2 in the example of FIG. 7, might occur. In this regard, FIG. 9A illustrates a portion 900 of the catheter device system 300 pressing into a portion of a tissue wall 902 of the bodily cavity in a state in which the electrogram 704b1 from the example of FIG. 7 is recorded in the hypothetical example of FIG. 9A. This pressing by the portion 900 of the catheter device system 300 into the portion of the tissue wall 902 creates a distortion in the tissue wall of the bodily cavity, which is recorded at a location indicated by location 706b1 associated with the electrogram 704b1. Such distortion is represented in FIG. 7 with hyphenated line 708, which branches into hyphenated line 708a and hyphenated line 708b, which encapsulate map distortion region 710. The transition from the state of FIG. 9A to the state of FIG. 9B illustrates a movement of the portion 900 of the catheter device system 300 in a direction away from the tissue wall 902, resulting in a release of the pressing tension in the tissue wall 902. With the tissue wall no longer substantially distorted in the state of FIG. 9B, electrogram 704b2 is recorded at a location associated with (or indicated by) location 706b2 in the hypothetical example of FIG. 9B. However, due to the lack of pressing-distortion in the tissue wall 902 in the state of FIG. 9B compared to the state of FIG. 9A, the location 706b2 is recorded as different than the location 706b1, even though, in this example, locations 706b1 and 706b2 are associated with a same relative location with respect to the tissue wall 902. By detecting the similarity of the corresponding electrograms 704b1 and 704b2, the data processing device system (e.g., 110, 310) is able to determine that the locations 706b1 and 706b2 actually represent the same locations on the tissue wall 902, resulting in the position alteration correction discussed above with respect to FIG. 8, according to some embodiments. The map correction produced by the position alteration of FIG. 8 compared to FIG. 7 is illustrated in FIG. 8 with the non-branching straight hyphenated line 808, which illustrates the stitching or closing up (removal) of the map distortion region 710 shown in FIG. 7, according to some embodiments.

In view of the above example with respect to FIGS. 9A and 9B, it can be seen that, at least when the supposed locations 706b1 and 706b2 in FIG. 7 are derived from position information provided by a navigation system (e.g., 342), errors may occur that may lead to an output that indicates that the two supposed locations 706b1 and 706b2 are different rather than being the same location, such as when the stretching and non-stretching states of the tissue wall occur as illustrated in FIGS. 9A and 9B. In this regard, such stretching and non-stretching states of the tissue wall may be detected or confirmed by detecting varying degrees of electrode-to-tissue contact experienced at the two supposed locations 706b1 and 706b2. Distortions in maps present at least from position information provided by a navigation system (e.g., 342) may also or alternatively be detected or confirmed at least by detecting (e.g., by the data processing device system (e.g., 110, 310)) changes in tissue or body characteristics occurring between successive samplings revealing tissue impedance changes.

As discussed above, some embodiments of the altering per some embodiments of block 510 in FIG. 5 may cause the resulting position set (e.g., location 806b in the example of FIG. 8) associated with the resulting electrogram set (e.g., electrogram 804b) to be a selected one or more of the locations associated with the compared electrograms (e.g., a selection of either location 706b1 or location 706b2 in the example of FIG. 7), for instance, in a state in which the data processing device system (e.g., 110, 310) is able to determine that such one or more of the locations is associated with a non-distorted or less-distorted state of the corresponding portion of the map of the bodily cavity. With reference to the examples of FIGS. 9A and 9B, the data processing device system (e.g., 110, 310) may be configured to determine that the state of FIG. 9A is associated with a higher-distortion state of the corresponding portion of the map of the bodily cavity compared to the state of FIG. 9B at least by evaluating the degrees of tissue contact exhibited by the corresponding transducers (e.g., 220, 306, 406) in the states of FIGS. 9A and 9B to determine that the state of FIG. 9A is a stretched (and distorted) state of the tissue wall 902, while the state of FIG. 9B is not or is less of a stretched (or distorted) state of the tissue wall 902. In such a case with the data processing device system (e.g., 110, 310) determining that the state of FIG. 9A is a stretched (distorted) state of the tissue wall 902 while the state of FIG. 9B is not a stretched (or distorted) state of the tissue wall 902, the data processing device system (e.g., 110, 310) may be configured to select the location 706b2 of the electrogram 704b2 as the correct position for the electrograms 704b1 and 704b2 for the altering performed in accordance with block 510 in FIG. 5, according to some embodiments.

In some embodiments, the first position information (accessed per block 504) is associated with a first portion of the bodily cavity (e.g., a first portion including location 706b1 in the example of FIG. 7), and the second position information (accessed per block 508) is associated with a second portion of the bodily cavity (e.g., a second portion including location 706b2 in the example of FIG. 7). In some embodiments, the data processing device system (e.g., 110, 310) is configured by the program at least to produce the map (e.g., corrected map 802 in the example of FIG. 8) at least by stitching at least part of a first submap (e.g., first submap 710a in map distortion region 710 shown in the example of FIG. 7) of a first part of the bodily cavity with at least part of a second submap (e.g., second submap 710b in map distortion region 710 shown in the example of FIG. 7) of a second part of the bodily cavity, the first submap of the first part of the bodily cavity mapping at least part of the first portion of the bodily cavity, and the second submap of the second part of the bodily cavity mapping at least part of the second portion of the bodily cavity. In some embodiments, the stitching utilizes the altered first position information, the altered second position information, or both the altered first position information and the altered second position information. For instance, referring to the examples of FIGS. 7 and 8, the altering per some embodiments of block 510 in FIG. 5, which results in the stitching illustrated by hyphenated lines 708, 808 in the transition between the states of FIGS. 7 and 8, utilizes the altered (e.g., corrected) location 806b to correct the map distortion region 710, such correction effecting such illustrated stitching, according to some embodiments.

In some embodiments, the data processing device system (e.g., 110, 310) may be configured by the program (e.g., per the comparison of some embodiments of block 510 in FIG. 5) at least to determine that the first position information (e.g., accessed per block 504) and the second position information (e.g., accessed per block 508) are associated with a same tissue region of the bodily cavity (e.g., in the example of FIG. 7, location 706b1 and location 706b2 are determined to be associated with a same tissue region, in some embodiments). In some embodiments, the comparison (e.g., per block 510) of the at least one electrogram in the first electrogram set and the at least one electrogram in the second electrogram set indicates that the first position information and the second position information are associated with a same tissue region of the bodily cavity (e.g., in the example of FIG. 7, location 706b1 and location 706b2 are determined to be associated with a same tissue region, in some embodiments). In some embodiments, the altering of the first position information, the second position information, or both the first position information and the second position information produces altered position information to indicate the same tissue region of the bodily cavity. For instance, in the examples of FIGS. 7 and 8, the location 806b may indicate or be represented by the altered position information, which indicates that the pre-corrected locations 706b1, 706b2 are now being treated as the same location 806b, according to some embodiments.

In some embodiments, the altered position information (e.g., represented by location 806b in the example of FIG. 8) indicates a resulting position associated with both the at least one electrogram (e.g., electrogram 704b1 in an example of FIG. 7) in the first electrogram set and the at least one electrogram (e.g., electrogram 704b2 in an example of FIG. 7) in the second electrogram set, the resulting position being other than (a) the position (e.g., location 706b1) associated with the at least one electrogram in the first electrogram set, (b) the position (e.g., location 706b2) associated with the at least one electrogram in the second electrogram set, or each of (a) and (b), the resulting position indicating a location on the same tissue region of the bodily cavity (e.g., location 806b indicating a location in a tissue region including locations 706b1, 706b2), according to some embodiments. In some embodiments, the data processing device system (e.g., 110, 310) may be configured by the program at least to exclude (c) at least part of the first position information (e.g., accessed per block 504), (d) at least part of the second position information (e.g., accessed per block 508), or both (c) and (d) in the altered position information. For example, with reference to the example of FIGS. 7 and 8, the altered position information may be represented by location 806b, the first position information may be represented by location 706b1, and the second position information may be represented by location 706b2. In such an example, the altered position information (e.g., indicating at least location 806b) may be deemed to exclude the first position information (e.g., indicating at least location 706b1) and the second position information (e.g., indicating at least location 706b2) at least because the altered position information represents a different location than both the first position information and the second position information. In some embodiments, the altered position information is deemed to exclude (c) at least part of the first position information (e.g., accessed per block 504), (d) at least part of the second position information (e.g., accessed per block 508), or both (c) and (d) in instances where the altered position information is not derived from (c), (d), or (c) and (d).

In some embodiments, the altering per block 510 in FIG. 5 is of both the first position information (e.g., accessed per block 504) and the second position information (e.g., accessed per block 508). For instance, in some embodiments where, e.g., the compared electrograms (e.g., compared per block 510) have moderate similarity (e.g., enough similarity to modify their originally supposed locations closer together, but not enough similarity to merge them into a single location), the data processing device system (e.g., 110, 310) may be configured to alter, e.g., in the memory device system (e.g., 130, 330), the contents of a data structure for each of the first position information (e.g., accessed per block 504) and the second position information (e.g., accessed per block 508) so that the compared electrograms have altered locations that are closer together, according to some embodiments.

In some embodiments, the altering of the first position information, the second position information, or both the first position information and the second position information includes altering the at least the position associated with the at least one electrogram in the first electrogram set, altering the at least the position associated with the at least one electrogram in the second electrogram set, or both altering the at least the position associated with the at least one electrogram in the first electrogram set and altering the at least the position associated with the at least one electrogram in the second electrogram set. For instance, with respect to the example of FIGS. 7 and 8, the resulting altered location 806b represents an adjustment of the location 706b1 associated with the electrogram 704b1 (an example of the first electrogram set), an adjustment of the location 706b2 associated with the electrogram 704b2 (an example of the second electrogram set), or an adjustment of the location 706b1 associated with the electrogram 704b1 and an adjustment of the location 706b2 associated with the electrogram 704b2, according to various embodiments.

In some embodiments, the comparison of the at least one electrogram in the first electrogram set and the at least one electrogram in the second electrogram set per some embodiments of block 510 may include determining a similarity metric indicating a degree of similarity between the at least one electrogram in the first electrogram set (e.g., electrogram 704b1 in the example of FIG. 7) and the at least one electrogram in the second electrogram set (e.g., electrogram 704b2 in the example of FIG. 7). In some embodiments, the similarity metric is calculated according to a Root Mean Square of Deviation (RMSD) calculation or a Root Mean Square Error (RMSE) calculation, where the at least one electrogram in the first electrogram set may be considered the observed values in the calculation and the at least one electrogram in the second electrogram set may be considered the predicted values in the calculation, or vice versa. For instance, in the case where each electrogram set is a single electrogram, the RMSD may be defined by equation (1), below,

$$RMSD = \sqrt{{\sum }_{n = 1}^{N} {( {x}_{n} - {y}_{n} )}^{2}}$$

where, xₙ and yₙ indicate the voltage values of each electrogram at discrete sample n, each with total length N. The above equation may be generalized to electrograms of differing length using methods, such as by restricting to overlapping portions or by zero-padding.

In some embodiments, the similarity metric is calculated according to a maximum cross-correlation. For instance, the maximum cross-correlation (MCC) may be defined by equation (2), below,

$$MCC = {max}_{- N \leq l \leq N} {\sum }_{n = - N}^{N} {x}_{n} * {y}_{n - l}$$

where, xₙ and yₙ indicate the voltage values of each electrogram at discrete sample n after zero-padding to be defined in the range of −N≤n≤N where N is the length of both electrograms prior to zero-padding and l is a relative offset (lag) applied between the two signals for comparison purposes. As above, this equation may be generalized to electrograms of differing length using methods, such as by restricting to overlapping portions or by zero-padding.

In some embodiments, the similarity metric is calculated according to a weighted combination of maximum cross-correlation and the corresponding time offset, where the corresponding time offset is defined by equation (3), below,

$${l}_{MCC} = {arg max}_{- N \leq l \leq N} {\sum }_{n = - N}^{N} {x}_{n} * {y}_{n - l}$$

In some embodiments, the similarity metric is calculated according to a normalized maximum cross-correlation. For instance, the normalized maximum cross-correlation (NMCC) may be defined by equation (4), below,

$$NMCC = {max}_{- N \leq l \leq N} \frac{{\sum }_{n = - N}^{N} {x}_{n} * {y}_{n - l}}{{σ}_{x} {σ}_{y}}$$

where σₓ and σ_{y} represent the standard deviation of each electrogram.

In some embodiments, the similarity metric is calculated according to a weighted combination of normalized maximum cross-correlation and the corresponding time offset, where the corresponding time offset is defined by equation (5), below,

$${l}_{NMCC} = {arg max}_{- N \leq l \leq N} \frac{{\sum }_{n = - N}^{N} {x}_{n} * {y}_{n - l}}{{σ}_{x} {σ}_{y}}$$

In some embodiments where each electrogram set includes multiple electrograms (i.e., in a comparison of groups of electrograms), the metric is calculated by assigning a pairwise correspondence between a plurality of electrograms in the first set and a plurality of electrograms in the second set, such that each pair contains one electrogram from the first set and one electrogram from the second set, and computing the mean value of each such pairwise comparison according to one of the above described metrics. In some embodiments, this correspondence may be determined through a nearest neighbor operation or by assigning correspondence between all pairs within a maximum distance. In some embodiments, the mean operation applied to pairwise comparisons is a weighted mean. In some embodiments the weights of the weighted mean are responsive to distances between the locations associated with the corresponding electrograms and/or the total number of pairs that include a particular electrogram. In this regard, in some embodiments, the data processing device system (e.g., 110, 310) may be configured by the program at least to determine the similarity metric based at least on a determination of a root mean square deviation between the at least one electrogram in the first electrogram set and the at least one electrogram in the second electrogram set. In some embodiments, the data processing device system (e.g., 110, 310) may be configured by the program at least to determine the similarity metric based at least on a determination of a cross-correlation between the at least one electrogram in the first electrogram set and the at least one electrogram in the second electrogram set. In some embodiments, the data processing device system (e.g., 110, 310) may be configured by the program at least to determine the similarity metric based at least on a weighted combination of a result of each of (a) a determination of a root mean square deviation between the at least one electrogram in the first electrogram set and the at least one electrogram in the second electrogram set, and (b) a determination of a cross-correlation between the at least one electrogram in the first electrogram set and the at least one electrogram in the second electrogram set.

In some embodiments, the degree of altering referred to in block 510 in FIG. 5 is proportional to the degree of similarity indicated by the determined similarity metric. In some embodiments, a higher degree of similarity indicated by the determined similarity metric results in a higher degree of the altering referred to in block 510 in FIG. 5. For instance, a higher degree of similarity indicated by the determined similarity metric may indicate a greater likelihood that the compared electrograms per some embodiments of block 510 represent a same location on the tissue surface of the bodily cavity and, therefore, may result in a greater shift in position for at least one of the locations associated with the compared electrograms, in comparison to a state in which those same electrograms were not as similar. In such a case in which those same electrograms were not as similar, but were still determined to have a sufficient degree of similarity, the data processing device system (e.g., 110, 310) may be configured to determine that, although the positions associated with the compared electrograms are not the same location on the tissue surface, but represent closer locations than indicated by their present supposed locations, and, therefore, their present supposed locations should be altered to be closer together albeit not to the same position, according to some embodiments.

In some embodiments, the comparison of the at least one electrogram in the first electrogram set and the at least one electrogram in the second electrogram set per some embodiments of block 510 in FIG. 5 is a comparison of a plurality of electrograms in the first electrogram set with a plurality of electrograms in the second electrogram set. For instance, although the above discussions with respect to the example of FIG. 7 focus on comparing electrogram 704b1 with electrogram 704b2, according to some embodiments, other embodiments (such as at least some of those described above with respect to equations (2)-(5)) may include a configuration of the data processing device system (e.g., 110, 310) to compare a plurality of electrograms in the first electrogram set with a plurality of electrograms in the second electrogram set. For example, with reference to FIG. 7, according to some embodiments, the data processing device system (e.g., 110, 310) may be configured to include the electrograms 704a and 704b1 in the first electrogram set and to include the electrograms 704b2 and 704c in the second electrogram set, such that the comparison referred to in block 510 includes a comparison of (a) electrograms 704a, 704b1 with (b) electrograms 704b2, 704c.

In some embodiments, a comparison of groups of electrograms such as this may be implemented at least by combining the electrograms in each group to result in a single electrogram for each group, such that the single electrograms are compared, according to some embodiments. In some embodiments, electrograms in a group of electrograms may be combined by averaging the electrograms in the group to form the resulting single electrogram. In some embodiments, electrograms in a group of electrograms may be combined by selecting a representative electrogram from the group of electrograms, such that the selected representative electrogram is determined to best represent the group of electrograms, e.g., by having the most common characteristics of the electrograms in the group.

In some embodiments, a comparison of groups of electrograms may be implemented at least by individually comparing all or a representative number of all permutations of combinations of individual electrograms across the groups of electrograms. In some of these embodiments, a similarity between the groups of electrograms may be determined by combining, such as averaging or some other form of combining, the individual similarity metrics from all or a representative number of the individual similarity metrics determined from the permutations of the combinations of the individual electrograms. For example, with respect to FIG. 7, assume that electrograms 704a, 704b1 are the first electrogram set and that electrograms 704b2, 704c are the second electrogram set. In such a case, in some embodiments, the data processing device system (e.g., 110, 310) may be configured to compare, per some embodiments of block 510, (1) electrogram 704a with electrogram 704b2, (2) electrogram 704a with electrogram 704c, (3) electrogram 704b1 with electrogram 704b2, and (4) electrogram 704b1 with electrogram 704c, thereby executing comparisons of all possible permutations of combinations of electrograms in the first electrogram set with electrograms in the second electrogram set. Such four comparisons (1)-(4) could result, in some embodiments, with four similarity metrics, which could be combined, e.g., by averaging, to result in a final similarity metric for the comparisons of the first and second electrogram sets. Such final similarity metric could then be utilized to determine whether altering should occur per some embodiments of block 510 in FIG. 5 and, if so, by what amount, according to various embodiments.

In some embodiments, when comparing groups of electrograms per some embodiments of block 510 in FIG. 5, the locations respectively associated with the electrograms in a group may be combined to determine a location associated with the group, and the determined location may be utilized for the altering of position information per some embodiments of block 510. For instance, with respect to FIG. 7, assume that electrograms 704a, 704b1 are the first electrogram set and that electrograms 704b2, 704c are the second electrogram set. In such a case, in some embodiments, the data processing device system (e.g., 110, 310) may be configured to combine the locations 706a and 706b1 for the first electrogram set and to combine the locations 706b1 and 706c for the second electrode set, according to some embodiments. Such combining, in some embodiments, may take the form of averaging of the positions. According to some embodiments, the combined positions for each respective group may then be utilized by the data processing device system (e.g., 110, 310) when determining the altered position information per some embodiments of block 510.

In this regard, in some embodiments, a comparison of a plurality of electrograms in the first electrogram set with a plurality of electrograms in the second electrogram set per some embodiments of block 510 may indicate that the first position information (e.g., from block 504) and the second position information (e.g., from block 508) are associated with a same tissue region of the bodily cavity, such that the altering of the first position information, the second position information, or both the first position information and the second position information per some embodiments of block 510 may produce altered position information to indicate the same tissue region of the bodily cavity. For instance, in a case in which the example of FIG. 7 involves a comparison of the group of electrograms 704a, 704b1 with the group of electrograms 704b2, 704c, the data processing device system (e.g., 110, 310) may be configured to determine that, due the high degree of similarity between electrograms 704b1 and 704b2, the group of electrograms 704a, 704b1 has sufficient similarity with the group of electrodes 704b2, 704c, and, therefore, that the first position information (e.g., which may indicate a region of space associated with locations 706a, 706b1) and the second position information (e.g., which may indicate a region of space associated with locations 706b2, 706c) are associated with a same tissue region of the bodily cavity, according to some embodiments. In such a case, the data processing device system (e.g., 110, 310) may be configured to alter the first position information, the second position information, or both the first position information and the second position information per some embodiments of block 510 to produce altered position information indicating the same tissue region of the bodily cavity, e.g., as described above at least with respect to FIG. 8.

Although the above discussions with respect to the example of FIG. 7 focus on comparing electrogram 704b1 with electrogram 704b2 or comparing the group of electrograms 704a, 704b1 with the group of electrograms 704b2, 704c, according to some embodiments, some embodiments may include a configuration of the data processing device system (e.g., 110, 310) to repeat block 510 in FIG. 5 to cycle through all or a subset of electrograms recorded by all transducers (e.g., 220, 306, 406) and comparing each set or subset thereof with all or a subset of all other electrograms, either individually or as subgroups, according to various embodiments. Such a cycling process may, for instance, allow the data processing device system (e.g., 110, 310) to determine and correct areas of distortion in all or some of the map of the bodily cavity, according to some embodiments.

According to some embodiments, methods 500 in FIG. 5 may include block 512 associated with computer-executable instructions (e.g., altering instructions provided by a program) configured to cause a data processing device system (e.g., 110, 310) to cause, via the input-output device system (e.g., 120, 320), display of a graphical representation of a map of at least part of a bodily cavity based at least on the altered first position information (e.g., altered per some embodiments of block 510), the altered second position information (e.g., altered per some embodiments of block 510), or both the altered first position information and the altered second position information (e.g., altered per some embodiments of block 510). With respect to FIG. 8, the corrected map 802 may represent or be a graphical representation of a map of at least part of a bodily cavity displayed based at least on an alteration of the location 706b1 (an example of the first position information), an alteration of the location 706b2 (an example of the second position information), or an alteration of both the location 706b1 and the location 706b2, according to some embodiments. Although the corrected map 802 has a relatively simple appearance for ease of illustration, such a map may take other forms including, but not limited to, a three-dimensional form like that of map 602 shown in FIG. 6, according to various embodiments.

In some embodiments, the data processing device system is configured by the program at least to produce the map, such that the produced map reduces distortion in a previous version of the map due at least to the altering. For instance, with respect to the examples of FIGS. 7 and 8, the corrected map 802 reduces distortion in map 702, which is the previous version of the corrected map 802, according to some embodiments.

While some of the embodiments disclosed above are described with examples of cardiac electrophysiological activity, the same or similar embodiments may be used in other bodily organs, for example, the brain.

While some of the embodiments disclosed above are described with examples of cardiac ablation, the same or similar embodiments may be used for ablating other bodily organs or any lumen or cavity into which the devices of the present invention may be introduced.

Subsets or combinations of various embodiments described above can provide further embodiments.

These and other changes can be made to the invention in light of the above-detailed description. For instance, although particular catheter device systems are illustrated in various figures such as FIGS. 2, 3A, 3B, 4, 6, 9A, and 9B merely for discussion purposes, various embodiments of the invention utilize other types of catheter device systems, e.g., that are able to record electrograms. In general, in the following claims, the terms used should not be construed to limit the invention to the specific embodiments disclosed in the specification and the claims but should be construed to include other transducer-based device systems including all medical treatment device systems and all medical diagnostic device systems in accordance with the claims. Accordingly, the invention is not limited by the disclosure, but instead its scope is to be determined entirely by the following claims.

What is claimed is:

1. A medical system comprising:
a data processing device system;
an input-output device system communicatively connected to the data processing device system, the input-output device system communicatively connectable to a plurality of electrodes located on a catheter of a catheter device system; and
a memory device system communicatively connected to the data processing device system and storing a program executable by the data processing device system, the data processing device system configured by the program at least to:
access first electrogram information indicating a first electrogram set;
access first position information indicating at least a position associated with at least one electrogram in the first electrogram set;
access second electrogram information indicating a second electrogram set;
access second position information indicating at least a position associated with at least one electrogram in the second electrogram set, the position associated with the at least one electrogram in the first electrogram set different than the position associated with the at least one electrogram in the second electrogram set;
alter, based at least on a comparison of the at least one electrogram in the first electrogram set and the at least one electrogram in the second electrogram set, the first position information, the second position information, or both the first position information and the second position information; and
cause, via the input-output device system, display of a visual representation of a map of at least part of a bodily cavity based at least on the altered first position information, the altered second position information, or both the altered first position information and the altered second position information.

2. The medical system of claim 1,
wherein the first position information is associated with a first portion of the bodily cavity,
wherein the second position information is associated with a second portion of the bodily cavity, and
wherein the data processing device system is configured by the program at least to produce the map at least by stitching at least part of a first submap of a first part of the bodily cavity with at least part of a second submap of a second part of the bodily cavity, the first submap of the first part of the bodily cavity mapping at least part of the first portion of the bodily cavity, and the second submap of the second part of the bodily cavity mapping at least part of the second portion of the bodily cavity, and
wherein the stitching utilizes the altered first position information, the altered second position information, or both the altered first position information and the altered second position information.

3. The medical system of claim 1,
wherein the data processing device system is configured by the program at least to determine that the first position information and the second position information are associated with a same tissue region of the bodily cavity, and
wherein the altering of the first position information, the second position information, or both the first position information and the second position information produces altered position information to indicate the same tissue region of the bodily cavity.

4. The medical system of claim 1,
wherein the comparison of the at least one electrogram in the first electrogram set and the at least one electrogram in the second electrogram set indicates that the first position information and the second position information are associated with a same tissue region of the bodily cavity, and
wherein the altering of the first position information, the second position information, or both the first position information and the second position information produces altered position information to indicate the same tissue region of the bodily cavity.

5. The medical system of claim 4, wherein the altered position information indicates a resulting position associated with both the at least one electrogram in the first electrogram set and the at least one electrogram in the second electrogram set, the resulting position being other than (a) the position associated with the at least one electrogram in the first electrogram set, (b) the position associated with the at least one electrogram in the second electrogram set, or each of (a) and (b), the resulting position indicating a location in the same tissue region of the bodily cavity.

6. The medical system of claim 5, wherein the data processing device system is configured by the program at least to exclude (c) at least part of the first position information, (d) at least part of the second position information, or both (c) and (d) in the altered position information.

7. The medical system of claim 1,
wherein the comparison of the at least one electrogram in the first electrogram set and the at least one electrogram in the second electrogram set is a comparison of a plurality of electrograms in the first electrogram set with a plurality of electrograms in the second electrogram set,
wherein the comparison of the plurality of electrograms in the first electrogram set with the plurality of electrograms in the second electrogram set indicates that the first position information and the second position information are associated with a same tissue region of the bodily cavity, and
wherein the altering of the first position information, the second position information, or both the first position information and the second position information produces altered position information to indicate the same tissue region of the bodily cavity.

8. The medical system of claim 1,
wherein the data processing device system is configured by the program at least to receive, via the input-output device system, first navigation information and second navigation information,
wherein the first position information is or is derived from the first navigation information, and
wherein the second position information is or is derived from the second navigation information.

9. The medical system of claim 8,
wherein the input-output device system is communicatively connected to a catheter navigation system, and
wherein the first navigation information and the second navigation information are received, via the input-output device system, from the catheter navigation system.

10. The medical system of claim 1, wherein the altering of the first position information, the second position information, or both the first position information and the second position information includes altering the at least the position associated with the at least one electrogram in the first electrogram set, altering the at least the position associated with the at least one electrogram in the second electrogram set, or both altering the at least the position associated with the at least one electrogram in the first electrogram set and altering the at least the position associated with the at least one electrogram in the second electrogram set.

11. The medical system of claim 1, wherein the comparison of the at least one electrogram in the first electrogram set and the at least one electrogram in the second electrogram set includes determining a similarity metric indicating a degree of similarity between the at least one electrogram in the first electrogram set and the at least one electrogram in the second electrogram set.

12. The medical system of claim 11, wherein a higher degree of similarity indicated by the determined similarity metric results in a greater degree of the altering.

13. The medical system of claim 1, wherein the data processing device system is configured by the program at least to produce the map, and wherein the produced map reduces distortion in a previous version of the map due at least to the altering.

14. The medical system of claim 1, wherein the input-output device system is communicatively connected to the catheter device system.

15. The medical system of claim 14, wherein the data processing device system is configured by the program at least to receive the first electrogram information from a first electrode set from the plurality of electrodes and to receive the second electrogram information from a second electrode set from the plurality of electrodes.

16. The medical system of claim 15, wherein the first electrode set comprises at least one electrode that is not in the second electrode set.

17. The medical system of claim 14, wherein at least some of the plurality of electrodes are configured to cause pulsed field ablation of tissue.

18. The medical system of claim 15, wherein at least one electrode from the first electrode set, the second electrode set, or both the first electrode set and the second electrode set is or are configured to cause pulsed field ablation of tissue.

19. The medical system of claim 1, wherein the position associated with the at least one electrogram in the first electrogram set is different than the position associated with the at least one electrogram in the second electrogram set.

20. The medical system of claim 1, wherein the first electrogram information indicates first electrophysiological information recorded by the catheter device system at a first position in the bodily cavity, and the second electrogram information indicates second electrophysiological information recorded by the catheter device system at a second position in the bodily cavity, the second position different than the first position.
